(19) United States
(12) Patent Application Publication
(10) Pub. No.: US 2010/0003679 A1
(43) Pub. Date: Jan. 7, 2010

(54) ASSESSMENT OF CELLULAR COMPOSITION AND FRACTIONAL VIABILITY AND USES THEREOF

(75) Inventors: Hirohito Ichii, North Bay Village, FL (US); Camillo Ricordi, Miami, FL (US)

Correspondence Address: ROPES & GRAY LLP, PATENT DOCKETING 39/361, 1211 AVENUE OF THE AMERICAS, NEW YORK, NY, 10036-8704, US

(73) Assignee: University of Miami, Miami, FL (US)

(21) Appl. No.: 12/308,234

(22) PCT Filed: Jun. 8, 2007
(86) PCT No.: PCT/US2007/070772

§ 371 Date: May 15, 2009

Related U.S. Application Data
(60) Provisional application No. 60/812,367, filed on Jun. 9, 2006.

(51) Int. Cl. G01N 1/30; C12Q 1/68

(52) U.S. Cl. 435/6; 435/40.5

(57) ABSTRACT

A method of assessing cellular composition and fractional viability that can be predictive of post-transplant cell potency and transplantation outcome, comprises identifying cellular composition and assessing cellular viability. This has particular importance in the field of tissue and cell transplantation, cell therapy and regenerative medicine, providing a method for tissue and cell characterization, viability and potency testing, that could be useful for the definition of product release criteria for research and clinical applications.

### FIELD OF THE INVENTION

This invention relates to the field of tissue and cell transplantation, cell therapy and regenerative medicine, providing a method for tissue and cell characterization, viability and potency testing, that could be useful for the definition of product release criteria for research and clinical applications.

### BACKGROUND

Recent improvements in islet isolation and immunosuppression have made transplantation of human islets a viable treatment for patients with type 1 diabetes mellitus (1-2). While selected centers have reported high rates of success (1-5), there have been reports of failures occurring in the very early post-transplant period. This disappointing observation could be related to the use of islet preparations of less than optimal quality (6-8). This problem is linked to the lack of reliable markers of islet potency to screen human islet preparations prior to transplantation.

Currently accepted product release criteria include viability measured by DNA-binding dye exclusion (9), islet cell purity based on dithizone (DTZ) staining (10-11), and in vitro glucose-stimulated insulin release (11-12). According to these pre-transplant criteria, islet preparations that failed to reverse diabetes were indistinguishable from those that resulted in excellent function. DNA-binding dye exclusion can only reveal cells that have lost membrane perm-selectivity. Additionally, this method does not identify apoptotic cells or determines whether dead cells preferentially belong to any given subset. Dithizone staining can only provide an estimate of endocrine cell content in islet preparations, but does not allow for the definition of β-cell content (11). A more predictive pre-transplant test is diabetes reversal in immunodeficient mice (11-13); however, several days are required for outcome assessment, making it an unpractical pre-transplant product release criteria.

There is, therefore, a critical need for alternative analytical methods to define islet cellular composition and fractional viability (including early events of apoptosis) before clinical transplantation. The Food and Drug Administration's Biological Response Modifiers Committee has recently underlined the need for developing such methods (14-15).

### SUMMARY

A novel method of analysis to precisely and objectively quantify cellular composition and fractional β-cell viability in human islets, is based on the use of Laser Scanning Cytometry (LSC) and cytofluorimetry. Analysis of human islet preparations with these techniques allows for the definition of β-cell mass and viability, and are important for potency testing of human islets before transplantation. This method can be utilized for any cell type, cells harvested from any organ, such as for example, heart, lungs, liver, skin, kidney, bone marrow, and the like.

In one preferred embodiment, the methods allow for the identification of cells that would have the highest success rate in transplantation in a patient or individual. The method is based on quantifying and identifying a particular cell type and the viability of such cells. For example, one of skill in the art may desire to isolate stem cells from a patient or individual for transplanting in the same patient or another individual.

In a preferred embodiment, a method for the assessment of β-cell content and viability in HICP allows for >90% prediction of diabetes reversal in immunodeficient mice.

In another preferred embodiment, a method of assessing cellular composition and fractional viability that can be predictive of post-transplant cell potency and transplantation outcome, comprises identifying cellular composition and assessing cellular viability comprising isolating cells from an organ, tissue; dissociating the organ or tissue into single cells; fixing, incubating with antibodies and/or staining of the single cells; subjecting one aliquot of cells to methods which identify cellular composition, such as, for example, laser scanning cytometry, immuno-histochemistry or electron microscopy; and, subjecting one aliquot of cells to flow cytometry; assessing cellular viability to predict transplantation outcome.

The cells can be stained with DNA and/or zinc binding dyes, 7-aminoactinomycin D (7-AAD), Fluorescein Diacetate, Ethidium Bromide or equivalent DNA binding stains.

In another preferred embodiment, the cells are further stained with mitochondrial stains to assess cellular viability. Examples of mitochondrial stains include, but not limited to: Newport Green PDX acetoxymethylether (NG); tetramethylrhodamine ethyl ester (TMRE), cyanine or xanthylium dyes.

In another preferred embodiment, the cells are detected with agents specific for apoptotic markers. Examples include, but not limited to: annexin V, TUNEL Stain, 7-amino-actinomycin D and Caspase substrates.

In another preferred embodiment, the cell composition comprises beta cells, alpha cells, acinar cells and ductal cells. Beta cells are identified as NG^{bright}TMRE⁺.

In a preferred embodiment, antibodies are specific for pancreatic cell markers and subsets thereof, comprising insulin, glucagon, somatostatin, pancreatic polypeptide, ductal cell markers, progenitor or stem cell markers, inflammatory or immune cell markers.

In another preferred embodiment, assessing cellular viability and predictive transplantation outcome comprises quantifying cellular composition and fractional beta-cell viability. Preferably, the cellular composition is quantified by measuring viable β-cell index, β-cell Mass/kg, Viable β-cell Mass/kg. The predictive transplantation outcome can be measured as: Diabetes Reversal Index=total islet equivalents (IEQ)×(% β-cell content in the islets)×(% non-apoptotic β-cells)/Insulin IU×10,000. Viable β-cell Equivalent Number/kg (vβEQ/kg)=[islet β-cell content×β-cell fractional viability×IEQ/kg] and compared to insulin reduction rate per kg, and insulin independence after islet infusion into a patient.

In another preferred embodiment a method of identifying cells suitable for transplantation comprises isolating cells from an organ, tissue or bodily fluids; identifying cellular composition; assessing viability of cells in the cellular composition; and, identifying cells suitable for transplantation. The specific cells can be identified by cell specific antigens (e.g. HLA antigens, surface molecules and the like), biomarkers, antibodies and functional assays (e.g. CTL assays, T-cell proliferation assays, insulin measurement etc.).

In another preferred embodiment, the cellular composition and viability are assessed by laser scanning cytometry and cytofluorimetry.

In another preferred embodiment, a method of identifying cell damage comprises isolating cells from an organ, tissue or bodily fluids; identifying cellular composition; assessing viability of cells in the cellular composition; and, identifying cell damage.

In another preferred embodiment, a method for identifying ductal cells and determining viability and/or potency, comprises: identifying cellular composition and assessing cellular viability comprising isolating cells from an organ, tissue; dissociating the organ or tissue into single cells; fixing, incubating with antibodies and/or staining of the single cells; subjecting one aliquot of cells to laser scanning cytometry, immuno-histochemistry or electron microscopy; and, subjecting one aliquot of cells to flow cytometry; assessing cellular viability and/or potency.

In one preferred embodiment, ductal cells are identified by pan-ductal membrane antibody, CA19-9 (human PDC CA19-9) and compared to that of CK19 using LSC/iCys.

In another preferred embodiment, identification of ductal cells is predictive for long term function.

In another preferred embodiment, a method for identifying of progenitor and/or stem cells, and determining their viability/potency, predictive of the regenerative potential and/or long term function, comprising: isolating cells from bone marrow or organ; dissociating the bone marrow or organ into single cells; fixing, incubating with antibodies and/or staining of the single cells; subjecting one aliquot of cells to laser scanning cytometry, immuno-histochemistry or electron microscopy; and, subjecting one aliquot of cells to flow cytometry; identifying of progenitor and/or stem cells, and determining their viability/potency, predictive of the regenerative potential and/or long term function.

In another preferred embodiment, the stem cells are identified by stem cell markers, for example Sca⁺, c-kit⁺.

In another preferred embodiment, a method of identifying and determining the viability/potency of inflammatory and immune cells predictive of the early loss of transplanted cells after infusion/implantation and/or predictive of the probability of acute and chronic rejection/recurrence of autoimmunity, comprising: isolating cells from organ, tissue or bodily fluid; obtaining single cells from the organ tissue, or bodily fluid; fixing, incubating with antibodies and/or staining of the single cells; subjecting one aliquot of cells to laser scanning cytometry, immuno-histochemistry or electron microscopy; and, subjecting one aliquot of cells to flow cytometry; and, identifying and determining the viability/potency of inflammatory and immune cells predictive of the early loss of transplanted cells after infusion/implantation and/or predictive of the probability of acute and chronic rejection/recurrence of autoimmunity.

In another preferred embodiment, wherein inflammatory and immune cells are identified by cell specific markers comprising: MCP-1, HLA Class I and II, CD80, CD86, CD40, CD40L, TGF-beta, interleukins, α, β, or γ-IFN, TNF, CD4, CD25, Foxp3, VEGF receptor-2(FLK-1), TRK (an NGF receptor), transferrin receptor, and annexin II (lipocortin 2), CD4, CD104, CD117, heat shock protein-27, tumor rejection antigen, glutathione-S transferase, peroxiredoxin 1, voltage-dependent-anion channel-2, protein kinase C substrate, phosphatase 2A inhibitor, esterase D, RNase A, initiation factor 5a, elongation factor 1-alpha, ribosomal protein S12, ribosomal protein large P1, ribosomal protein large P2, transcription factor BTF 3a, annexin I, destrin, myosin light chain, lactate dehydrogenase A, glycerolaldehyde-3-P dehydrogenase, citrate synthetase, transketolase, P-glycerolmutase, aldo-keto reductase 7(A2), alpha-amylase inhibitor CM3, enoyl-CoA hydratase, proteosome subunit alpha-4, stromal derived factor 1 (SDF-1), MCP-1, MIP-1a, MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, MAdCAM-1, α4 integrins, α5 integrins, beta defensins 3 and 4. Apoptic cell markers used for identifying apoptic cells comprise, annexin V, TUNEL Stain, 7-amino-actinomycin D and Caspase substrates.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A-1C Analysis of cellular composition in human islets by LSC. An aliquot of approximately 100 IEQ was dissociated to obtain single cell suspension. Single cells were divided into four individual aliquots, and each aliquot was stained with one of the indicated antibodies. The appropriate fluorochrome-conjugated secondary antibody was subsequently added. FIG. 1A is a scan of a photograph whereby the desired area to be scanned was visually located using the microscope connected to the instrument, and the examined area was mapped using the Wincyte software. FIG. 1B: For removal of aggregated cells from further analysis, single cells were identified based on DAPI fluorescence emission area. FIG. 1C: Analysis with LCS allowed for the computation of the percentage of positive cells (cytoplasm stained in green, nucleus in blue by DAPI) in each preparation, by recording positive and negative cells (the latter revealed by blue nuclear staining in the absence of green cytoplasmic staining). The instrument software graphically depicts the results in the form of the plots shown here, where both percentage of positive events and events' intensity are shown. The data shown is representative of more than 60 human islet preparations.

FIGS. 2A-2B are graphs showing beta cell content variability in individual human islet preparation. FIG. 2A is a graph showing the relation between β-cell percentage in whole islet preparations and purity, the latter assessed by DTZ staining. Beta-cell percentage was calculated as fraction of insulin positive cells over all cells (not only the endocrine subsets). Results were obtained by analyzing more than 60 preparations. FIG. 2B is a graph showing percentages of cells belonging to the indicated endocrine subsets were calculated and expressed as fraction over endocrine cells only, excluding non-endocrine cells from computation. Results were obtained by analysis of over 60 preparations.

FIG. 3 shows an LSC analysis of β-cell content in viable and dead islet cells. Two aliquots of islets from the same preparation were cultured either in condition of hypoxia/starvation (Pellet+, left panels), or in conventional condition (Pellet−, right panels). The immunofluorescence images of hormone-specific staining with anti-insulin (green) and anti-glucagon (red) antibodies (upper panels), and the quantification of insulin positive cells by LSC (middle panels) revealed no differences, while analysis of viability, as assessed by 7-AAD staining with FACS (lower panels) revealed, as expected, a dramatic difference, with 78% dead (7-AAD⁺) cells in the experimental aliquot versus 13% dead cells in the control aliquot. This comparative analysis clearly shows that LSC, while providing precise and objective quantification on β-cell composition, does not allow analysis of β-cell viability. The results shown are representative of at least 5 independent experiments.

FIGS. 4A-4B show the identification of human beta cells by the zinc-binding dye Newport Green. FIG. 4A shows single cells were stained with NG and 7-AAD. Single cell suspensions were analyzed by FACS and sorted into NG^{bright}, and NG^{dim/negative} subsets after exclusion of dead cells by 7-AAD. The left panel of the figure shows the NG staining pattern prior to sorting, the middle panels show the staining pattern of the individual sorted subsets, clearly showing high enrichment for bright and dim/negative cells respectively. Immunofluorescence analysis of the two sorted subsets (right upper panel: NG^{bright}; right lower panel: NG^{dim/negative}) shows high enrichment for β-cells (stained in green) in the NG^{bright} subset (approximately 90%) and high depletion of β-cells in the NG^{dim/negative} subset (approximately 10%). Glucagon cells are stained in red, and other cells (non-α, non-β) only stain with DAPI (blue). The results shown are representative of at least ten individual experiments. FIG. 4B is a graph showing the comparison of β-cell composition analysis by LSC and FACS. Twenty-seven human islet preparations were analyzed by LSC and by FACS to ascertain the percentage of β-cells revealed by either technique, and to compare them. It clearly appears that a tight correlation exists in all individual preparations in the results generated by either analytical technique. Since analysis by LSC includes dead cells, while analysis by FACS is performed after exclusion of dead cells, the tight correlation observed in this study strongly argues against selective exclusion of β-cells from FACS analysis by exclusion of dead cells.

FIG. 5 shows the assessment of apoptosis in human beta and non-β-cell subsets. The figure summarizes the analytical methodology utilized to assess viability and apoptosis of β-cells and, complementarily, of non-β-cells. After dispersion of human islets into single cell suspensions, cells are stained with 7-AAD, NG and TMRE. The percentage of 7-AAD⁺ cells (dead cells) is recorded (upper panel), and further analysis is performed after their exclusion (gating out). NG^{bright} and NG^{dim/negative} cells are visualized, and β-cell composition is recorded (second panel from top). The two subsets identified by NG staining are then individually analyzed for the relative percentages of TMRE⁺ and TMRE⁻ cells (two lower panels).

FIG. 6 shows the comparative analysis of cell viability, β-cell apoptosis and in vivo islet function. Islet aliquots were either cultured in conventional conditions (upper panels), or cultured in conditions leading to hypoxia/starvation (pellet) for 6 hour (middle panels) of 18 hours (lower panels). Analysis of cell viability by conventional means was performed on whole cells by 7-AAD staining (left panels). Analysis of β-cell apoptosis was performed by analysis of TMRE staining on NG^{bright} cells (vertical middle panels). Analysis of in vivo function was performed by transplantation in diabetic immunodeficient rodents (right panels). The figure shows that a 6-hr culture in hypoxia/starvation resulted in an increase in the percentage of apoptotic β-cells (reduced % of TMRE⁺, NG^{bright} cells), and in loss of function in vivo, while analysis of cell viability by conventional means showed no difference, when compared to the control. When 18-hr hypoxia/starvation was used, also non-selective analysis of cell viability reveals the detrimental effect of such treatment. This data argues in favor of a higher sensitivity of our novel analytical method as a predictive test of in vivo function, when compared to non-specific viability assays based on DNA-binding dye exclusion. Results shown are representative of at least 5 independent experiments, where 3 mice per condition were transplanted.

FIGS. 7A-7D shoe the analysis of β-cell apoptosis after delivery of noxious stimuli. Islets were incubated in the presence or absence of the indicated noxious stimuli: sodium nitroprusside (SNP), an NO donor (FIG. 7A); H₂O₂ (FIG. 7B); IL1-β (FIG. 7C); and IL1-β, TNF-α, and IFN-γ (FIG. 7D). Apoptosis was analyzed by TMRE staining in the beta cell (NG^{bright}) subset. Incubation of islet cells in any of the four conditions resulted in increased apoptosis in β-cells, suggesting a selective pro-apoptotic effect of the studied compounds on insulin-producing β-cells. Additionally, these results show that our method of islet β-cells viability could be widely applicable also in experimental conditions that arguably mimic events occurring in vivo at the transplant site. Data is representative of at least five independent experiments in each experimental condition.

FIG. 8 show β-cell-specific analysis of viability/apoptosis in islet preparations with different purity. Islet aliquots with different degrees of purity from more than 60 preparations were assessed with our method. Representative data comparing three preparations (I, II, and III) with different degrees of purity is shown. After gating the 7AAD⁻ cell population, percentages of TMRE⁺ cells in total living cells (top panels), of NG^{bright} cells (β-cells; middle panels), and of TMRE⁺ cells within the NG^{bright} cells (β-cells fractional viability; bottom panels) were analyzed. These data suggests that assessment of overall viability in islet cells may not represent an adequate estimate of β-cell viability: islet preparation II would have led to overestimation of β-cells viability, while islet preparation III would have led to underestimation. Data is representative of more than 100 independent experiments.

FIGS. 9A-9B show the predictive value of β-cell content/viability on in vivo islet function. Aliquots of 2,000 IEQ from 24 individual islet preparations were transplanted to each of 82 diabetic immunodeficient mice. FIG. 9A shows a plot of all transplanted preparations where β-cell content (%) and β-cell fractional viability (%) are recorded and related to transplant success. There is a clear trend to increased success rate when β-cell content and fractional viability are higher. In FIG. 9B, the product of β-cell content (%) and β-cell fractional viability (%) was calculated (β-cell viability index) and related to in vivo assessment of islet potency. It is clear that the higher the β-cell viability index, the better the transplant outcome. Statistical analysis also suggests that β-cell fractional viability is an independent predictor that is significantly positively associated with success rate.

FIG. 10 is a graph showing the predictive value of β-cell content/viability on in vivo islet function.

FIG. 11 is a graph showing the predictive value of viable β-cell index on human transplanted islet function.

FIG. 12 is a graph showing insulin reduction rate (>60%) by viable β-cell mass.

FIG. 13 is a graph showing the prediction in insulin reduction rate (>60%) by viable β-cell mass/kg.

FIG. 14 is a graph showing the prediction in insulin independence.

FIG. 15 is a graph showing the prediction in insulin independence by viable β-cell mass/kg.

FIG. 16 is a graph showing the prediction in insulin independence by diabetes reversal index.

FIGS. 17A-17F show the phenotypic analysis of PDC in human islet preparation by LSC/iCys. An aliquot of islet preparations was dissociated to obtain single cell suspensions. Fixed cells were stained with anti-CK19, amylase, glucagon, somatostatin and insulin antibodies. The appropriate fluorochrome-conjugated secondary antibody was subsequently added. FIG. 17A is a plot showing that single cells were identified based on DAPI fluorescence emission area, while aggregated cells were excluded from further analysis. FIG. 17B is a plot showing the LSC/iCys analysis of the double staining with CK19 and amylase revealed five cell subsets based on the intensity of fluorescence. FIG. 17C is a scan of a photograph showing that cells from every subpopulation were visualized directly in the LSC/iCys by re-localization to confirm regular morphology. Double staining with anti-CK19 was performed to help identifying the phenotype of CK19^{dull} and Amylase⁻ population. The results showed CK19^{dull} expression was absent in β-cells (FIG. 17F) but present in α-cells (FIG. 17E) and δ-cells (FIG. 17F). The data shown is representative of five independent human islet preparations.

FIGS. 18A-18C show the analysis of CA19-9 expression in PDC revealed the variability in individual human islet preparations. The expression of the pan-ductal membrane antibody for human PDC CA19-9 was compared to that of CK19 using LSC/iCys. FIG. 18A is a plot showing that there was a tight correlation observed between the percentage of CK19^{high} and CA19-9⁺ populations (n=34). FIG. 18B is a plot showing the human islet preparations tested, 5% ( 8/161) showed lack of CA19-9 expression even though a substanitial amount of PDC was measured by CK19. FIG. 18C is a plot showing the analysis of 203 fractions from 106 independent human islet preparations were examined to evaluate PDC content using anti-CA19-9 antibody on dissociated cells by FACS. A negative correlation between the percentage of CA19-9⁺ and islet purity assessed by DTZ was observed.

FIGS. 19A-19E are plots showing the simultaneous assessment of β-cells and PDC viability by FACS. Schematics of the analytical methodology utilized to assess simultaneously apoptosis of β-cells and PDC. After dissociation of human islets, single cell suspensions were stained with 7-AAD, NG, CA19-9 and TMRE. The percentage of 7-AAD⁺ cells (dead cells) is recorded (FIG. 19A), and further analysis is performed after their exclusion (gating out). The two subsets identified by NG and CA19-9 staining (FIG. 19B) are then individually analyzed for the relative percentages of CA19-9⁺ cells (PDC; FIG. 19C), CA19-9-NG⁻ cells (other cells; FIG. 19D) and NG⁺ cells (β-cells; FIG. 19E) expressing TMRE⁺ (viable cells) and TMRE⁻ (apoptotic cells).

FIGS. 20A-20D show the analysis of δ-cells and PDC apoptosis after delivery of noxious stimuli. Islet cells were incubated in the presence or absence of noxious stimuli before assessment of viability in dissociated cells. Islet cell viability following hypoxic/starving conditions (compaction of islet preparations)(FIG. 20A), H₂O₂ (FIG. 20B); Nitric Oxide donor SNAP (FIG. 20C); and cytokine cocktail (IL1-β, TNF-α and IFN-γ)(FIG. 20D). Apoptosis was analyzed by TMRE staining in the β-cell and ductal cell subsets. Incubation of islet cells in any of the four conditions resulted in increased apoptosis in both β-cells and PDC populations, suggesting a selective pro-apoptotic effect of the studied compounds on both cell subsets. Additionally, these results showed that ductal cells were more resistant against the noxious conditions, such as H₂O₂, NO and cytokines than β-cells. Data is representative of at least five independent experiments for each experimental condition.

FIGS. 21A-21C are graphs showing the analysis of β- and PDC-specific viability/apoptosis in islet fractions with different densities. FIG. 21A shows the analysis of 202 islet fractions showed no correlation between β- and PDC-specific viability. FIG. 21B shows representative data comparing three islet cell fractions with different degrees of purity collected from layers with increasing density: after gating the 7AAD⁻ cell population, the percentages of TMRE⁺ cells in NG^{bright} (β-cells) or CA19-9⁺ cells (PDC) were analyzed. FIG. 21C shows islet aliquots with different degrees of purity collected from high, medium and low-density layers were assessed by FACS. Although β-cell viability in medium and high-density fractions were significantly high and low when compared to low density fractions, the increase of PDC viability was observed as the density of fraction increased (lower purity).

FIGS. 22A-22B are graphs showing the functional analysis of PDC obtained from different density fractions. To evaluate the function of PDC from low or high density purification fractions of the same human islet preparation, we sorted PDC using anti-CA19-9 antibody. The sorted PDC were cultured for 24 hours before collection of supernatant and of the cells for the measurement of cytokine/chemokine and TF production, respectively. FIG. 22A shows cytokine/chemokine production in the PDC sorted from high density were higher than that of low-density fractions. FIG. 22B shows a significant reduction in terms of TF production was observed in PDC sorted form high density fraction, when compared to low density fractions. Data is representative of four independent human islet cell preparations.

### DETAILED DESCRIPTION

Methods for assessing the probability of a success rate in transplantation of cells or organs in patients.

The instant invention has many advantages over current methodologies to evaluate islet cell viability as they are largely based on tests that assess the exclusion of DNA-binding dyes. While these tests identify cells that have lost selective membrane permeability, they do not allow identification of apoptotic cells, which do not yet stain with DNA-binding dyes. Furthermore, current methods of analysis do not discriminate between cell subsets in the preparation and, in particular, they do not allow for selectively defining β-cell viability.

For these reasons we have developed novel methods for the specific assessment of β-cell content and viability in human islets based on cellular composition analysis through Laser Scanning Cytometry (LSC) coupled with identification of β-cell-specific apoptosis at the mitochondrial level.

### Definitions

The present section provides definitions of the terms used in the present invention in order to facilitate a better understanding of the invention.

As used herein, the singular forms “a”, “an” and “the” include plural referents unless the context clearly dictates otherwise.

The term “detectably label” is used to herein to refer to any substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert (e.g., from non-light absorbing to light absorbing molecules, or from non-fluorescent to fluorescent molecules). Analysis can be performed using any of a number of commonly used platforms, including multiparameter flow cytometry, immunofluorescent microscopy, laser scanning cytometry, bright field base image analysis, capillary volumetry, spectral imaging analysis, manual cell analysis, CellSpotter™ analysis, CellTracks™ analysis, and automated cell analysis.

As used herein, the term “specifically reactive” or “specifically binds to” or “specific for” when used in reference to an antibody refers to the discriminatory binding of the antibody to the indicated target polypeptide. For such binding to be discriminating, the antibody will not substantially cross react with other polypeptides. Specific reactivity can include binding properties such as binding specificity, binding affinity and binding avidity. For example, an antibody can bind a target polypeptide with a binding affinity (Kd) of about 10⁻⁴ M or more, 10⁻⁶ M or more, 10⁻⁷ M or more, 10⁻⁸ M or more, 10⁻⁹ M or more, or 10⁻¹⁰ M or more. Several methods for detecting or measuring antibody binding are known in the art and disclosed herein.

A “stem cell” is a relatively undifferentiated cell that can be induced to proliferate and that can produce progeny that subsequently differentiate into one or more mature cell types, while also retaining one or more cells with parental developmental potential. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype.

“Progenitor cells” have a cellular phenotype that is more primitive (i.e., is at an earlier step along a developmental pathway or progression than is a fully differentiated cell). Often, progenitor cells also have significant or very high proliferative potential. Progenitor cells may give rise to multiple distinct differentiated cell types or to a single differentiated cell type, depending on the developmental pathway and on the environment in which the cells develop and differentiate. Like stem cells, it is possible that cells that begin as progenitor cells might proceed toward a differentiated phenotype, but then “reverse” and re-express the progenitor cell phenotype.

The term “subject,” or “patient” as used herein, means a human or non-human animal, including but not limited to mammals such as a dog, cat, horse, cow, pig, sheep, goat, chicken, primate, rat, and mouse.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference.

### Cell Composition Quantification and Viability

In one embodiment, the method of assessing cellular composition and fractional viability that can be predictive of post-transplant cell potency and transplantation outcome, comprises identifying cellular composition and assessing cellular viability comprising isolating cells from an organ, tissue; dissociating the organ or tissue into single cells; fixing, incubating with antibodies and/or staining of the single cells; subjecting one aliquot of cells to methods which identify cellular composition, such as, for example, laser scanning cytometry, immuno-histochemistry or electron microscopy; and, subjecting one aliquot of cells to flow cytometry; assessing cellular viability to predict transplantation outcome.

In laser scanning cytometry (LSC), a laser beam scans a sample on a fixed medium such as a slide, and the position of the cells and each cell figures are simultaneously determined and recorded. Details regarding methods to use a LSC are well known in references in the art, such as Clatch et al. (1998); U.S. Pat. Nos. 5,427,910; 5,793,969; and 5,885,840, each herein incorporated by reference.

Construction and use of laser scanning cytometers has been described by Kamentsky and Kamentsky (Cytometry, 12:381-387, 1991) and in U.S. Pat. Nos. 4,647,531 and 5,072,382, the disclosures of which are incorporated herein by reference thereto.

In summary, a laser scanning cytometer such as the LSC™ cytometer, available from CompuCyte Corp., scans cell specimens, on a microscope slide positioned on a microscope stage, with a laser beam which is oscillating in the Y direction of the microscope stage. Voltage levels from optical detectors are synchronized and digitized to produce a raster of values from captured light. Subsequently, the microscope stage is advanced in the X direction, and the scan digitization is repeated. The cycle is repeated until a two dimensional (x-y) array of measurements is acquired. This two dimensional array is segmented by image processing techniques, and features for cells or objects of interest on the specimen are extracted and stored in a list mode data file.

One of the parameters measured by the LSC is forward light scatter. In this mode of operation, a focused laser beam passes through the specimen slide and laser scattered light is intercepted by a blocker bar before reaching a photodiode detector located beneath the blocker bar. In the reference position where there is no object in the laser beam path, the laser light is entirely prevented from reaching the detector, and the output of the detector is a low (zero) voltage signal. When a cell or other object is in the path of the laser beam, laser light is diverted from its original path, and is scattered over a range of angles (i.e., forward light scattering). A portion of this scattered laser light bypasses the blocker bar and strikes the face of the detector which provides an output signal characteristic of the particular way the light is blocked and scattered.

The output signal from the detector increases proportionally relative to the amount of light scatter. The resulting two dimensional memory array image appears as a dark field image with a black background, with the objects of interest appearing as a generally undefined bright image.

The software for LSC allows multiple different geographic regions for automatic scanning of a sample. The data obtained from the analysis are collected and stored within a computer file. Values for each of three fluorescence channels, including green, orange and long red, are obtained. A scattergram of y position versus x position maps the location of the cells on a slide.

In a preferred embodiment, analysis of clinical islet transplantation preparations, predict functional performance. These results can be correlated with in vivo analysis of islet potency in immunodeficient rodents.

The invention provides methods for quantifying and analyzing cellular compositions and viability of cells in tissues; organs, such as, for example, skin, heart, lung, bone marrow, kidney, liver pancreas and the like; bodily fluids such as for example, blood, blood plasma, serum, and other bodily fluids such as urine, effusions (including pleural effusions, pericardial effusions, and joint effusions), ascites, saliva, cerebrospinal fluid, cervical secretions, amniotic fluid, gastrointestinal secretions, sputum and bronchial secretions, breast fluid, synovial fluid, fluid from cysts, and tissue lavages, from an animal, most preferably a human.

The invention further provides methods for detecting, identifying, evaluating, monitoring, or providing a prognosis for transplantation therapy for diseases requiring a transplantation.

The methods of the invention comprise the steps of isolating cells from an organ, tissue, bodily fluid and dissociating the organ or tissue into single cells. In the case of obtaining cells from bodily fluids, cells may be extracted, purified, isolated, concentrated, separated, or labeled from any bodily fluid, including but not limited to whole blood, plasma, serum, urine, effusions, ascitic fluid, saliva, cerebrospinal fluid, cervical secretions, vaginal secretions, endometrial secretions, gastrointestinal fluids and secretions including fluids and secretions from the stomach, pancreas, liver, gallbladder, small intestines, and colon, bronchial secretions including sputum, breast fluid or secretions, or washings or lavages.

In the case of, for example, cells from blood, the blood may be drawn by routine venipuncture, or by finger-stick or capillary stick or from an indwelling venous access device such as a venous catheter. In a preferred embodiment, the blood is drawn by venipuncture with from 1-100 milliliters of blood obtained, although lesser amounts or greater amounts are acceptable. The blood can be kept on ice until use or processing. Preferably, within 1-3 hours of drawing the blood, plasma or serum is separated from the cellular fraction by centrifugation of blood, for example at 1100×g for 10 minutes at 4° C. When using plasma, the blood is not permitted to coagulate prior to separation of the cellular and acellular components.

Once the cells have been obtained, the samples are prepared for fixing, incubating with antibodies and/or staining of the single cells. The antibodies are preferably specific for desired cellular antigens, biomarkers etc, of the type of cell that one of skill in the art is to identify.

In one embodiment, the cells of interest are β-cells of the pancreas.

The pancreatic cells are isolated using methods know in the art. An exemplary method is described in the Examples section which follow. Briefly, the dispersed cells were fixed on glass slides with 2.5% paraformaldehyde. After permeabilization with 1% saponin for 15 min, cells were incubated with Protein Block for 30 min, to reduce non-specific binding. After washing in Optimax Wash Buffer, cells were incubated for 1 hour with the following antibodies: mouse monoclonal antibody to insulin (1:100), rabbit polyclonal antibody to somatostatin (1:100), from Neo Markers (Fremont, Calif.); mouse monoclonal antibody to glucagon (1:500; Sigma, St. Louis, Mo.); undiluted rabbit polyclonal antibody to pancreatic polypeptide (PP; Bio-Genex, San Ramon, Calif.). After washing, samples were incubated with either goat anti-mouse (Alexa Fluor 488 goat anti-mouse IgG, 1:200 dilution) or goat anti-rabbit (Alexa Fluor 488 goat anti-rabbit IgG, 1:200 dilution) antibodies, both from Molecular Probes (Eugene, Oreg.). Omission of the primary antibody served as negative control. After washing, 4′,6-diamidino-2-phenylindole (DAPI) was applied to stain cell nuclei. Samples were analyzed using a LSC (CompuCyte, Cambridge, Mass.).

In one preferred embodiment, antibodies are specific for ductal cells, acinar cells, dendritic cells, macrophages, T and B cells, endothelial cells, progenitor and stem cells, proinflammatory and immunogenicity markers such as, but not limited to tissue factor, MCP-1, HLA Class I and II, CD80, CD86, CD40, CD40L, TGF-beta, interleukins, e.g. IL-10, alpha-IFN, beta-IFN, gamma-IFN, TNF, CD4, CD25, Foxp3.

Examples of markers for stem cells include but not limited to: VEGF receptor-2 (FLK-1), TRK (an NGF receptor), transferrin receptor, and annexin II (lipocortin 2). CD4, CD104, CD117, heat shock protein-27, tumor rejection antigen, glutathione-S transferase, peroxiredoxin 1, voltage-dependent-anion channel-2, protein kinase C substrate, phosphatase 2A inhibitor, esterase D, RNase A, initiation factor 5a, elongation factor 1-alpha, ribosomal protein S12, ribosomal protein large P1, ribosomal protein large P2, transcription factor BTF 3a, annexin I, destrin, myosin light chain, lactate dehydrogenase A, glycerolaldehyde-3-P dehydrogenase, citrate synthetase, transketolase, P-glycerolmutase, aldo-keto reductase 7(A2), alpha-amylase inhibitor CM3, enoyl-CoA hydratase, and proteosome subunit alpha-4, Sca, c-kit.

In another preferred embodiment, the anti-inflammatory marker, include, but not limited to stromal derived factor 1 (SDF-1), MCP-1, MIP-1a, MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, MAdCAM-1, α4 integrins, α5 integrins, beta defensins 3 and 4.

Examples of apoptosis markers include but not limited to Annexin V, TUNEL Stain, 7-amino-actinomycin D and Caspase substrates. Other examples include detecting mRNA and/or protein markers of apoptosis; i.e., molecules known to be increased or decreased in apoptotic cells. Such markers are well known in the art. For instance, markers known to be increased in apoptotic tissues include, but are not limited to: caspases, annexin, DNAse I, DNAse II, NUC 18/cyclophilin, transglutaminase, Fas, FasL, p53, Diva, Bak, Bcl-X₉, Bik, Bim, Bad, Bid, Egl-1, and Bax, to name a few. Markers known to be decreased in apoptotic tissues include, but are not limited to, Bcl2, Bcl-X_{L}, Mcl-1 and CED-9.

Examples of immune and inflammatory cells, include for example, granulocytes, T cells, B cells, and monocytes. Cells are labeled with commercially-available antibody reagents and analyzed by cytometry. For example, granulocytes are identified using antibodies for the cell surface molecules CD15 and CD16. Within the granulocyte population, CD89 expression is quantified using antibodies for CD89. Both the relative number of granulocytes expressing CD89 and the intensity of expression of CD89 can be counted. CD4 T cells are identified using antibodies to CD4. CD38 expression on CD4 T cells is quantified using antibodies to CD38. Monocytes are identified using CD14 monoclonal antibodies, and B cells are identified using CD20 antibodies. Finally, HLA class II antigens are quantified using antibodies to HLA-PAN, HLA-DR, and HLA-DQ; and CD62L is identified using antibodies to CD62L.

In one preferred embodiment, the cells for transplantation are stem cells. The source of stem cells may be any natural or non-natural mixture of cells that contains stem cells. The source may be derived from an embryonic mammal, or from the post-natal mammal. One source of cells is the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

Either before or after the crude cell populations are purified as described above, the population of stem cells may be further concentrated by methods known in the art. For example, the stem cells can be enriched by positive selection for one or more antigens characteristic of stem cells. Such antigens include, for example, FLK-1, CD34, and AC133. For example, human stem cells may be pre-purified or post-purified by means of an anti-CD34 antibody, such as the anti-My-10 monoclonal antibody described by Civin in U.S. Pat. No. 5,130,144. The hybridoma cell line that expresses the anti-My monoclonal antibody is available from the American Type Culture Collection, 12301 Parklawn Drive, Rockville, Md. 20852, USA. Some additional sources of antibodies capable of selecting CD34⁺ cells include AMAC, Westbrook, Me.; Coulter, Hialea, Fla.; and Becton Dickinson, Mountain View, Calif. CD34⁺ cells may also be isolated by means of comparable antibodies, which may be produced by methods known in the art, such as those described by Civin in U.S. Pat. No. 5,130,144.

In addition, or as an alternative to, the enrichment with anti-CD34 antibodies, populations of stem cells may also be further enriched with anti-Sca antibodies; with the AC 133 antibodies described by Yin et al., Blood 90, 5002-5112 (1997) and by Miraglia et al., Blood, 90, 50135021 (1997). The AC133 antibodies may be prepared in accordance with Yin et al.; ibid, or purchased from Miltenyi Biotec.

Appropriate markers or antigens for detecting bone marrow derived cells (BMDC) are polypeptides or nucleic acids not normally found in tissues outside of the bone marrow. Examples of such markers include, but are not limited to, Flk-1 (Swissprot: locus VGR2_HUMAN, accession P35968), Sca-1 (Swissprot: locus ICE3_HUMAN, accession P42574), Thy-1 (Swissprot: locus THY1_HUMAN, accession P04216), Patched (Accession NP—000255.1 GI:4506247), CXCR (NP—003458.1 GI:4503175), survivin (Swissprot: locus BIR5_HUMAN, accession 015392), and the human homolog of mouse nucleostatin (NP—705775.1 GI:23956324) polypeptides and nucleic acids encoding all or a portion of these proteins.

In a preferred embodiment, laser based cytometric methods are used allowing for fluorescence-based quantitative measurements on tissue sections or other cellular preparations at single-cell level. An optics/electronics unit coupled to an argon and HeNe laser repeatedly scans along a line as the surface is moved past it on a computer-controlled motorized stage of an fluorescent microscope.

In another preferred embodiment, the method provided herein detects damage mediated by different noxious conditions, including ischemia/hypoxia, H₂O₂, NO, IL-1β and cytokine cocktails (25-27). This suggests that the method is sensitive enough to be of assistance in the detection of islet cell damage possibly resulting from different conditions related to donor brain death, pancreas procurement and preservation, as well as islet processing.

Besides endocrine cells, such as beta and alpha cells, the method could be used to characterize and assess viability/potency of non-endocrine cell subpopulations present in cell products, including ductal cells, acinar cells, progenitor and stem cells, inflammatory and immune cells.

The methods provided herein, have a wide range of applicability, such as for example, identification of cytotoxic effects of drugs on cells for use in identifying novel therapeutic agents. For example, certain embodiments of the present invention provide fluorescent dye flow cytometric detection systems, e.g., a three or four color fluorescent dye flow cytometric detection system, useful for monitoring the outcome of cell-mediated cytotoxicity studies, e.g., in a single container, e.g., a test tube. The dyes and probe components can possess cell membrane permeability characteristics that allow the end user to accurately detect the membrane and apoptotic-associated changes of the cells resulting, e.g., from testing pharmaceutical compounds, viability of cells for use in transplantation. The method can be configured to contain: (1) a fluorescent dye capable of staining a cell population, e.g., a membrane stain; (2) a second fluorescent dye capable of detecting dead and membrane-compromised dying cells, i.e., a vital stain; and (3) a third fluorescent dye bound to a membrane permeant caspase inhibitor probe capable of detecting early apoptotic cells. The fluorescent dyes should typically emit at different wavelengths that can be differentiated by flow cytometric instruments.

In one configuration of the invention, the amine reactive, green fluorescing dye, carboxyfluorescein diacetate succinimidyl ester (CFSE) is used as the membrane stain. CFSE emits at 517 nm and binds with amine groups. In certain embodiments, membrane compromised dead and dying cells are identified using the red emitting vital stain, 7-aminoactinomycin D (7-AAD). This DNA binding dye emits at 647 nm, allowing for the use of a third fluorescent probe emitting in the orange wavelength region. Early apoptotic cells resulting, e.g., from effector cell pharmaceutical reagents, can be detected by the use of fluorescence labeled inhibitors of caspases (FLICA) probes (see, e.g., U.S. Patent Application Pub. No. 2005/0136492). These fluorescent membrane permeant probes penetrate the cell membrane of live cells and covalently bind to active caspase enzymes in apoptotic cells. Sulforhodamine B, coupled to the fluoromethyl ketone (FMK) labeled tri-peptide caspase inhibitor sequence, VAD, yielded a poly caspase specific apoptosis detection probe (SR-VAD-FMK) emitting in the orange fluorescence region (586 nm) of the spectrum.

In certain embodiments, the invention can be used as a method of the analysis of effector cells, or donor cells, may be stained with a membrane stain (e.g., CFSE) rather than staining the target cell population or recipient cell population. This separation allows for the analysis of cytolytic activity effects on the effector or donor cells when stained with the remaining reagents after incubation with the target or recipient cells in the case of transplantation rejection.

The invention may also be used to assess the cytolytic effects of pharmaceutical reagents, therapeutics and/or radiation treatments on specific cell populations. For example, cells may be stained with a membrane stain (e.g., CFSE), then be incubated with the drug, receive radiation treatment or be incubated with other cells. Final analysis is made after adding the vital stain and apoptosis detection probe.

Certain embodiments of the invention include the use of four fluorescent reagents. The four reagents typically emit at different wavelengths, allowing multiplexing. The first reagent is a membrane stain and is used to identify a population of cells. The second reagent is a vital stain and is used to identify necrotic or late-stage apoptotic cells that have a compromised cell membrane. The third reagent is a cell permeant probe used to detect early apoptosis. The fourth reagent is a mitochondrial probe. Each should fluoresce at a different wavelength. Not all assay units contain all four reagent types.

Membrane Stains: The membrane stain is typically a detectable membrane stain, e.g., that can be used to detect a preselected population of cells. The membrane stain may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides).

The membrane stain is typically a stain that stains cell membranes, e.g., a cell permeant fluorescent dye with an active group that will form a covalent bond to proteins within the cell membrane, and as a result, be retained in the cell. The active groups are typically succinimidyl esters that bind with primary amines, or a methyl chloride that binds with free thiols, or a methyl bromide that binds with free thiols. Thus, certain membrane stains are thiol-reactive stains and certain membrane stains are amine-reactive stains. The dye may be fluorescent at all times or it may contain one or more acetate groups and become fluorescent when the membrane permeant probe enters the cell and esterase hydrolysis removes the acetate groups. In one embodiment, the membrane stain is used to stain the target and/or recipient cells (or to stain donor cells), and incubated.

Certain embodiments of the invention provide methods and kits that contain at least one membrane stain to stain the cells, e.g., a preselected population of cells. In one embodiment of the invention, the amine reactive, green fluorescing dye, carboxyfluorescein diacetate succinimidyl ester (CFSE) is used as the membrane cell stain. CFSE has an optimal excitation at 475 nm and emits at 517 nm and binds with amine groups.

In another embodiment, the orange fluorescing dye, Cell Tracker Orange, is used as the membrane cell stain. This is a thiol reactive dye and will react with thiol groups in cell membrane and cytoplasmic proteins. Cell Tracker Orange has an optimal excitation at 541 nm and emits at 565 nm.

Other membrane stains include, but are not limited to, Cell Tracker Blue CMF₂HC, Cell Tracker Blue CMHC, Cell Tracker Blue CMAC, BODIPY 493/503 Methylbromide, SNARF-1 SE or BODIPY 630/650 Methylbromide (BODIPY Far Red).

Vital Stains: The vital stain is typically a detectable vital stain, e.g., that can be used to detect a preselected population of cells. The vital stain may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides).

In certain embodiments of the invention, at least one stain is used as a vital stain to detect dead or necrotic cells, e.g., in a preselected population of cells. The vital stain can be a cell-impermeant DNA stain. This stain will enter membrane-compromised cells that have either died or are in very late stages of apoptosis, as the reagent will no longer be excluded from those cells. When this stain binds to or intercalates with DNA, it becomes detectable, e.g., fluorescent. In certain embodiments, the vital stain is added after the cells, e.g., effector and/or target cells or donor and/or recipient cells, are incubated, or pharmaceutical treatments or radiation treatments are made to the cells.

In one embodiment of the invention, 7-aminoactinomycin D (7-AAD) is used as the vital stain. 7-AAD is excited at 546 nm and emits at 647 nm when it is intercalated with the DNA.

In another embodiment of the invention, one can use other vital stains, such as DNA binding stains, that are not cell permeant such as, but not limited to, TO-PRO-3, TO-PRO-5, SYTOX Blue, SYTOX Green or SYTOX Orange.

Apoptosis Detection Probes: The apoptosis detection probe is typically a detectable apoptosis detection probe, e.g., that can be used to detect a preselected population of cells. The apoptosis detection probe may include any group that can be detected, e.g., by analytical means. For example, suitable groups may be detectable by fluorescence spectroscopy, fluorescence microscopy, confocal fluorescence microscopy, fluorescence image analysis, flow cytometry, laser scanning cytometry, plate multi-well fluorescence reader, or a scintillation counter. Thus, some suitable groups include florescent labels (e.g., fluorescein, rhodamines, Cy dyes, Bodipys, sulforhodamine 101, Quantum Dots, phycobiliproteins, etc.) and radionuclides (e.g., metallic radionuclides and non-metallic radionuclides).

Certain embodiments of the invention involve the use of at least one apoptosis detection probe that is cell permeant and capable of detecting cells in the early stages of apoptosis through later stages of apoptosis, e.g., in a preselected population of cells. In certain embodiments, a caspase affinity labeling probe is used as the apoptosis detection probe. This probe may be any agent capable of permeating the cell membrane and selectively binding, in a covalent manner, to one or more active caspases and facilitating their detection. In certain embodiments of the invention, the cell permeant apoptosis detection probe can be added after the effector and/or target cells, or donor and/or recipient cells are incubated, or pharmaceutical treatments or radiation treatments are made. Examples of apoptosis detection probes Poly-Caspase Fluorescent Label-D-FMK Poly-Caspase Fluorescent Label-VD-FMK Poly-Caspase Fluorescent Label-VAD-FMK Caspase-1 Fluorescent Label-YVAD-FMK Caspase-2 Fluorescent Label-VDVAD-FMK Caspase-3 and 7 Fluorescent Label-DEVD-FMK Caspases-4 and 5 Fluorescent Label-WEHD-FMK Caspase-6 Fluorescent Label-VEID-FMK Caspase-8 Fluorescent Label-LETD-FMK, or IFETD Caspase-9 Fluorescent Label-LEHD-FMK Caspase-10 Fluorescent Label-AEVD-FMK, or LELD Caspase-13 Fluorescent Label-LEED-FMK

For example, such probes include fluorescent labels (e.g., fluorescein derivatives, sulforhodamine derivatives, Cy dye derivatives, BODIPY derivatives, coumarin derivatives, Quantum Dots, or any fluorescent dye that can be attached to an amino group directly or by linkers).

Other caspase affinity labeling probes may contain the same labels and a 1 to 5 amino acid sequence, but utilize an aldehyde modification of the aspartic terminal carboxyl group (HCOO), a chloromethyl ketone group (CH₂Cl), an acyloxy reactive group ((COO)O—Ar, where Ar is [2,6-(CF₃)₂]benzoate and various derivative of same, or an aza-peptide epoxide modification of the aspartic acid (U.S. Patent Publication No. US 2004/0048327), or an aza-peptide Michael acceptor.

DNA Binding Stains or Intercalating Agents: The intercalating agent or compound useful for nucleic acid, e.g., DNA binding is an agent or moiety capable of insertion between stacked base pairs in the nucleic acid double helix. Examples of nucleic acid intercalating agents are well known in the art and any of them without limitations can be used in the presently claimed invention.

The terms “intercalating moiety” or “intercalator” are known in the art to refer to those compounds capable of non-covalent insertion between the base pairs of a nucleic acid duplex and are specific in this regard only to double-stranded (ds) portions of nucleic acid structures including those portions of single-stranded nucleic acids which have formed base pairs, such as in “hairpin loops”. The nucleic acid structures can be dsDNA, dsRNA or DNA-RNA hybrids. The term “intercalating agent or intercalator” is also used to describe the insertion of planar aromatic or heteroaromatic compounds between adjacent base pairs of double stranded DNA (dsDNA), or in some cases dsRNA.

DNA intercalating agents utilizing ethidium bromide have been used in various DNA analytical procedures. The intercalating agents are characterized by their tendency to intercalate specifically to double stranded nucleic acid such as double stranded DNA or RNA. Some intercalating agents have in their molecules a flat intercalating group such as phenyl group, which intercalates between the base pairs of the double stranded nucleic acid, whereby binding to the double stranded nucleic acid. Most of the intercalating agents are optically active and some of them are used in quantification of nucleic acids. Certain intercalating agents exhibit electrode response. Therefore, determination of physical change, especially optical or electrochemical change, may serve to detect the intercalating agents bound to a double stranded nucleic acid.

Electrochemically or optically active intercalating agents are, but are not limited to, ethidium, ethidium bromide, acridine, aminoacridine, acridine orange, proflavin, ellipticine, actinomycin D, daunomycin, mitomycin C, HOECHST 33342, HOECHST 33258, aclarubicin, DAPI, Adriamycin, pirarubicin, actinomycin, tris(phenanthroline)zinc salt, tris(phenanthroline)ruthenium salt, tris(phenantroline)cobalt salt, di(phenanthroline)zinc salt, di(phenanthroline)ruthenium salt, di(phenanthroline)cobalt salt, bipyridine platinum salt, terpyridine platinum salt, phenanthroline platinum salt, tris(bipyridyl)zinc salt, tris(bipyridyl)ruthenium salt, tris(bipyridyl)cobalt salt, di(bipyridyl)zinc salt, di(bipyridyl)ruthenium salt, di(bipyridyl)cobalt salt, and the like.

Intercalating agents which exhibit electrochemiluminescence may also be employed. Such intercalating agents are, but are not limited to, for example, luminol, lucigenin, pyrene, diphenylanthracene rubrene and acridinium derivatives. The electrochemiluminescene of the intercalating agents listed above may be enhanced by the enhancers such as luciferin derivatives such as firefly luciferin and dihydroluciferin, phenols such as phenyl phenol and chlorophenol as well as naphthols.

When these intercalating agents are further bound with the substances which generate signals capable of being detected directly or indirectly, higher detection sensitivity can be obtained by determining the signals combined with the signals from the intercalating agents. These substances which generate signals capable of being detected directly or indirectly include, for example, haptens such as biotin, trinitrobenzene sulfonic acid and dinitrobenzene sulfonic acid, fluorescent substances such as fluorescein isothiocyanate (FITC), phycocyanin and rhodamine, luminescent substances such as luminol, lucigenin and acridium ester derivatives as well as electrode active substances such as ferrocene and viologen. When using the substance from which the signal cannot be directly detected, such as the haptens listed above, enzyme-labeled anti-hapten antibodies such as enzyme-labeled avidin are used to determine the optical parameters such as absorbance, fluorescene, luminescene, quenching, circular dichroism and fluorescene polarization or, electrode activity is determined, whereby indirectly detecting the gene. Although one molecule of these substances is usually bound to one molecule of an intercalating agent, several molecules of these substances may be bound to one molecule of the intercalating agent, whereby enhancing the sensitivity. A number of agents have been described for labeling nucleic acids, whether probe or target, for facilitating detection of target nucleic acid. Suitable labels may provide signals detectable by fluorescence, radioactivity, colorimetry, X-ray diffraction or absorption, magnetism or enzymatic activity, and include, for example, fluorophores, chromophores, radioactive isotopes, enzymes, and ligands having specific binding partners. All are useful herein.

Fluorescent dyes are also suitable for detecting nucleic acids. For example, ethidium bromide is an intercalating agent that displays increased fluorescence when bound to double stranded DNA rather than when in free solution. Ethidium bromide can be used to detect both single and double stranded nucleic acids, although the affinity of ethidium bromide for single stranded nucleic acid is relatively low. Ethidium bromide is routinely used to detect nucleic acids following gel electrophoresis.

The intercalating agent useful for DNA binding or detecting nucleic acids is an agent or moiety capable of insertion between stacked base pairs in the nucleic acid double helix. Intercalating agents such as ethidium homodimer and ethidium bromide fluoresce more intensely when intercalated into double stranded DNA than when bound to single stranded DNA, RNA, or in solution.

Conventional electroconductive threading intercalators have a structure comprising a core portion of a naphthalene-diimide cyclic group, a pair of linker portions each of which is attached to each of the two ends of the core portion, and a pair of electroconductive ferrocene moieties each of which is attached to the other end of each linker. The ferrocene moiety has an oxidation-reduction activity and a conjugated system in which electrons freely move. Makino et al. in U.S. Pat. No. 6,368,807 teach an improved electrochemical detection procedure at a low electric potential applied to the electrode using an improved electroconductive threading intercalator compound.

Over the last decade, studies using rhodium intercalators containing phenanthrenequinone-diimine (phi) ligands displayed tight DNA binding by preferential intercalation, some with affinities and specificities approaching DNA-binding proteins as taught by Barton et al. in U.S. Pat. No. 6,221,586.

Gjerde, et al., in U.S. Pat. No. 6,210,885 describes reversible DNA-binding dyes as useful to enhance the detection of double stranded DNA. The term “reversible DNA-binding dye” is used to include DNA intercalator dyes and DNA groove binding dyes. As defined, a “DNA intercalator dye” is a generally planar, aromatic, ring-shaped chromophore molecule which binds to DNA in a reversible, non-covalent fashion, by insertion between the base pairs of the double helix. The term “DNA groove binding dye” is defined herein to mean those chromophore molecules which reversibly bind by direct interaction with the edges of base pairs in either of the grooves (major or minor) of nucleic acids. These dyes are included in the group comprising non-intercalative DNA binding agents. Non-limiting examples of DNA groove binding dyes include Netropsin (N′-(2-amidinoethyl)-4-(2-guanidinoacetamido)-1,1′-dimethyl-N,4-′-bi[pyrrole-2-carboxamide]) (Sigma), Hoechst dye no. 33258 (Bisbenzimide, B-2261, Sigma), Hoechst dye no. 33342, (Bisbenzimide, B2261, Sigma), and Hoechst dye no. 2495 (Benzoxanthene yellow, B-9761, Sigma). Preferred reversible DNA-binding dyes in the present invention include fluorescent dyes. Non-limiting examples of reversible DNA-binding dyes include PICO GREEN (P-7581, Molecular Probes), ethidium bromide (E-8751, Sigma), propidium iodide (P-4170, Sigma), Acridine orange (A-6014, Sigma), 7-aminoactinomycin D (A-1310, Molecular Probes), cyanine dyes (e.g., TOTO, YOYO, BOBO, and POPO), SYTO, SYBR Green I, SYBR Green II, SYBR DX, OliGreen, CyQuant GR, SYTOX Green, SYTO9, SYTO10, SYTO17, SYBR14, FUN-1, DEAD Red, Hexidium Iodide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, and LDS 751. Numerous reversible DNA-binding dyes are described in Handbook of Fluorescent Probes and Research Chemicals, Ch. 8.1 (1997) (Molecular Probes, Inc.); European Patent Application No. EP 0 634 640 A1; Canadian Patent No. CA 2,119,126; and in the following U.S. Pat. Nos. 5,410,030; 5,321,130; 5,432,134; 5,445,946; 4,716,905 (which publications are incorporated by reference herein).

Intercalators according to Acevedo, et al., U.S. Pat. No. 6,060,592, generally include non-carcinogenic, polycyclic aromatic hydrocarbons or heterocyclic moieties capable of intercalating between base pairs formed by a hybrid oligonucleotide/RNA target sequence duplex. Intercalators can include naphthalene, anthracene, phenanthrene, benzonaphthalene, fluorene, carbazole, acridine, pyrene, anthraquinone, quinoline, phenylquinoline, xanthene or 2,7-diazaanthracene groups. Other intercalators believed to be useful are described by Denny, Anti-Cancer Drug Design 1989, 4, 241. Another intercalator is the ligand 6-[[[9-[[6-(4-nitrobenzamido)hexyl]amino]acridin-4-yl]carbonyl]-amino]hex-anoylpentafluorophenyl ester.

Bieniarz, et al., in U.S. Pat. No. 6,015,902, in U.S. Pat. No. 5,599,932, in U.S. Pat. No. 5,582,984 and in U.S. Pat. No. 5,808,077, discuss compounds which have been found to provide enhanced fluorescence when bound to a DNA molecule within a fluorescent flow cytometry environment which is about eight to ten times brighter in fluorescence than “bis” structure conventional intercalating agents and other known intercalating agents utilized in flow cytometry environment.

In other embodiments, minor groove dyes or binders can be used. A minor groove binder is a molecule that binds within the minor groove of the double stranded deoxyribonucleic acid (DNA). Most minor groove binding compounds have a strong preference for A-T (adenine and thymine) rich regions of the B form of double stranded DNA.

Minor groove binders have also been extensively described in the art and all can be used with this invention. Examples of minor groove binders include Hoechst 33258, CDPI₁ 3, netropsin, and distamycin. Linkers between a label and the PNA/DNA chimera can be an amide bond, e.g. where the active ester form of a label is coupled with an amino group of the chimera. Also, linkers can comprise alkyldiyl, aryldiyl, or one or more ethyleneoxy units (U.S. Pat. No. 6,469,151). U.S. Pat. No. 6,482,843 describes the minor groove binder plicamycin and U.S. Pat. No. 6,451,588 describes the minor groove binder CDPI₃. U.S. Pat. No. 5,801,155 describes covalently linked oligonucleotide minor groove binder conjugates. The U.S. Pat. No. 5,801,155 patent describes that naturally occurring compounds such as netropsin, distamycin and lexitropsin, mithramycin, chromomycin A₃, olivomycin, anthramycin, sibiromycin, as well as further related antibiotics and synthetic derivatives are minor groove binders. Certain bisquartemary ammonium heterocyclic compounds, diarylamidines such as pentamidine, stilbamidine and berenil, CC-1065 and related pyrroloindole and indole polypeptides, Hoechst 33258, 4′-6-diamidino-2-phenylindole (DAPI) as well as a number of oligopeptides consisting of naturally occurring or synthetic amino acids are also minor groove binder compounds.

In addition to the molecular structure which causes minor groove binding, the minor groove binder moiety may also carry additional functions, as long as those functions do not interfere with minor groove binding ability. For example a reporter group, which makes the minor groove binder readily detectable by color, UV spectrum or other readily discernible physical or chemical characteristics, may be covalently attached to the minor groove binder moiety. An example for such a reporter group is a diazobenzene function which in the example of a preferred embodiment is attached to a carbonyl function of the minor groove binder through a —HN(CH₂)ₘCOO(CH₂)ₘS(CH₂)ₘ— bridge. Again, the reporter group or other like function carried by the minor groove binder can also be conceptualized as part of the minor groove binder moiety itself.

Non-intercalating minor groove DNA-binding molecules include, but are not limited to the following: distamycin A, netropsin, mithramycin, chromomycin and oligomycin, which are used as antitumor agents and antibiotics; and synthetic antitumor agents such as berenil, phthalanilides, aromatic bisguanylhydrazones and bisquaternary ammonium heterocycles. Non-intercalating DNA-binding molecules vary greatly in structure: for example, the netropsin-distamycin series are oligopeptides compared to the diarylamidines berenil and stilbamidine.

A third category of DNA-binding molecules includes molecules that have both groove-binding and intercalating properties. DNA-binding molecules that have both intercalating and minor groove binding properties include actinomycin D, echinomycin, triostin A, and luzopeptin. In general, these molecules have one or two planar polycyclic moieties and one or two cyclic oligopeptides. Luzopeptins, for instance, contain two substituted quinoline chromophores linked by a cyclic decadepsipeptide. They are closely related to the quinoxaline family, which includes echinomycin and triostin A, although they luzopeptins have ten amino acids in the cyclic peptide, while the quinoxaline family members have eight amino acids.

In addition to the major classes of DNA-binding molecules, there are also some small inorganic molecules, such as cobalt hexamine, which is known to induce Z-DNA formation in regions that contain repetitive GC sequences. Another example is cisplatin, cis-di-amminedichloroplatinum(II), which is a widely used anticancer therapeutic. Cisplatin forms a covalent intrastrand crosslink between the N7 atoms of adjacent guanosines.

Distamycin is a member of a family of non-intercalating minor groove DNA-binding oligopeptides that are composed of repeating units of N-methylpyrrole groups. Distamycin has 3 N-methylpyrrole groups. Daunomycin is a member of an entirely different class of DNA-binding molecules, the anthracycline antibiotics, that bind to DNA via intercalation. Examples of homopolymers would be bis-distamycin, the dimer of distamycin, a molecule containing 6 N-methylpyrrole groups or tris-distamycin, the trimer of distamycin, a molecule containing 9 N-methylpyrrole groups. Heteropolymers are molecules composed of different types of DNA-binding subunits; for example, compounds composed of a distamycin molecule linked to a daunomycin molecule or a distamycin molecule linked to two daunomycin molecules. The term “oligomeric” is being used to describe molecules comprised of linked subunits each of which may be smaller than the parent compound.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### Examples

### Materials and Methods

Pancreas Preservation and Human Islet Isolation: Human islet isolations were performed at the Human Cell Processing Facility of the University of Miami School of Medicine from human pancreata preserved with either preoxygenated (30 min) two-layer perfluorocarbon/University of Wisconsin solution (PFC/UW) or with UW alone (16). Islets were isolated using a modification of the automated method (1,12,17). A total of sixty-two consecutive islet preparations were analyzed using the methods described below.

Cell dissociation: Single cell suspensions were obtained from human islets by incubating aliquots of approximately 1,000-1,500 islet equivalents (IEQ) (11,12) in 1 ml Accutase solution (Innovative Cell Technologies, Inc, San Diego, Calif.) at 37° C. for 10-15 min, followed by gentle pipetting. This method was selected after comparison with other techniques (trypsin-based and non-enzymatic buffers), as it provided consistent cellular dispersion, high yield, and transcurable viability loss.

Analysis of Cellular Composition by Immunofluorescence: Dispersed cells were fixed on glass slides with 2.5% paraformaldehyde (Electron Microscopy Sciences, Washington, Pa.). After permeabilization with 1% saponin for 15 min, cells were incubated with Protein Block (Bio-Genex, San Ramon, Calif.) for 30 min, to reduce non-specific binding. After washing in Optimax Wash Buffer (Bio-Genex, San Ramon, Calif.), cells were incubated for 1 hour with the following antibodies: mouse monoclonal antibody to insulin (1:100), rabbit polyclonal antibody to somatostatin (1:100), from Neo Markers (Fremont, Calif.); mouse monoclonal antibody to glucagon (1:500; Sigma, St. Louis, Mo.); undiluted rabbit polyclonal antibody to pancreatic polypeptide (PP; Bio-Genex, San Ramon, Calif.). After washing, samples were incubated with either goat anti-mouse (Alexa Fluor 488 goat anti-mouse IgG, 1:200 dilution) or goat anti-rabbit (Alexa Fluor 488 goat anti-rabbit IgG, 1:200 dilution) antibodies, both from Molecular Probes (Eugene, Oreg.). Omission of the primary antibody served as negative control. After washing, 4′,6-diamidino-2-phenylindole (DAPI) was applied to stain cell nuclei. Samples were analyzed using a LSC (CompuCyte, Cambridge, Mass.).

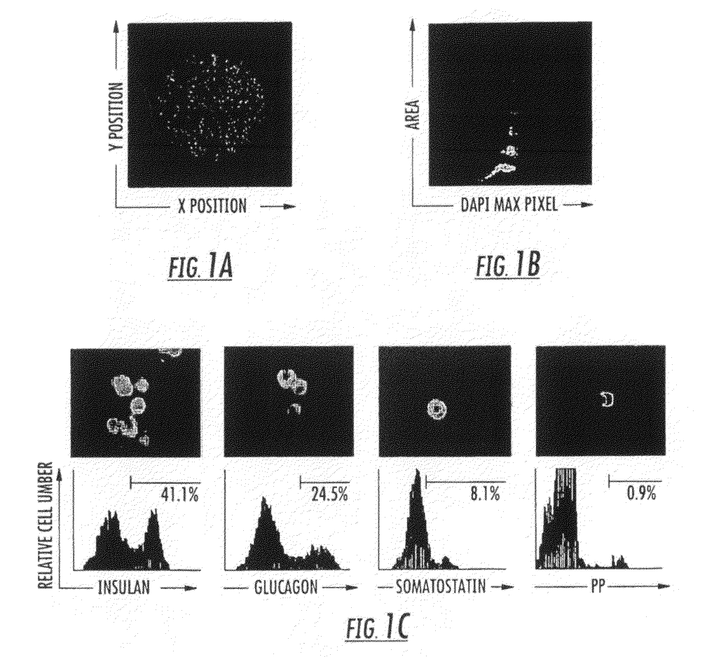

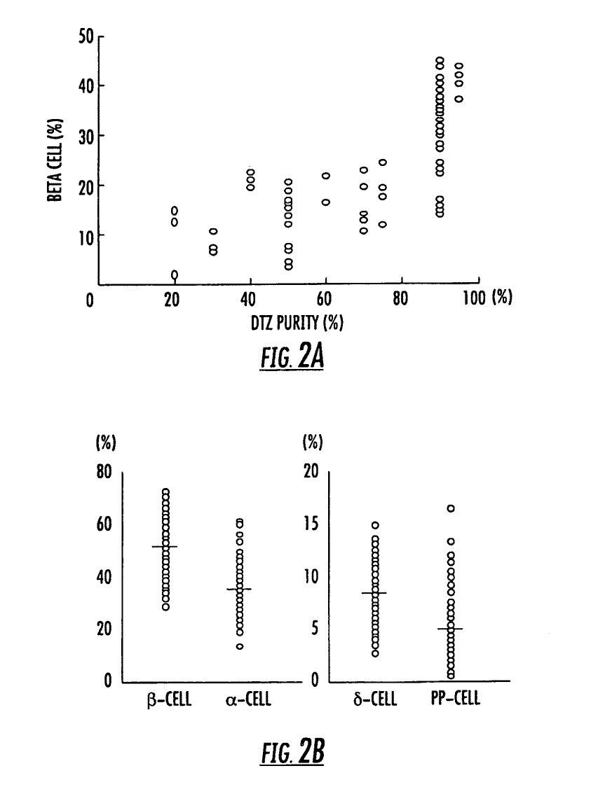

LSC analysis and data display: Data acquisition and analysis were performed using LSC at the Imaging Core of the Diabetes Research Institute. The LSC allows for fluorescence-based quantitative measurements on tissue sections or other cellular preparations at single-cell level. An optics/electronics unit coupled to an argon and HeNe laser repeatedly scans along a line as the surface is moved past it on a computer-controlled motorized stage of an Olympus BX50 fluorescent microscope (Melville, N.Y.). LSC was used to determine the percentage of each hormone-positive cell on the glass slides. The area to be scanned was visually located, and mapped using the Wincyte software (CompuCyte)(FIG. 1A). Slides were scanned at 40×, and nuclei were contoured using UV laser and DAPI detector. Each hormone-positive event was recorded using the argon laser and green (Alexa-488) detector. The following data were acquired: area, x position, y position, fluorescence integral, and maximal intensity for all channels. Single cells were identified and gated according to the DAPI staining area (FIG. 1B). Fluorescence intensity was recorded on a histogram (FIG. 1C). Cells from every subpopulation were visualized directly in the LSC by relocalization to confirm regular morphology. A minimum of 10,000 cells were acquired and analyzed for each sample.

Analysis of cell composition on whole islet sections: Isolated islets were formalin fixed and paraffin embedded. Sections were cut and stained with hormone-specific antibodies (insulin, glucagon and somatostatin). Nuclei were counterstained with DAPI. Sections were analyzed with a confocal microscope (Zeiss LMS 520). Five independent human islet preparations were analyzed and compared to the cellular composition of dissociated islet cells in the same preparations assessed by LSC. At least 5 randomly selected fields per preparation were assessed. Beta, α- and δ-cells were counted and percentages were calculated by using the DAPI staining to number all islet cells.

Assessment of β-cell content in dissociated islets: Beta-cell content within islets was calculated based on the analysis of immunostaining for endocrine markers by LSC using the formula:

$$\frac{β - cell \%}{( β + α + δ + PP - cells ) \%} \times 100.$$

Determination of fractional β-cell viability. For assessment of apoptosis, single cell suspensions were incubated with 1 μM Newport Green PDX acetoxymethylether (NG; Molecular Probes) and 100 ng/ml of tetramethylrhodamine ethyl ester (TMRE; Molecular Probes) for 30 min at 37° C. in PBS without Ca²⁺ and Mg²⁺. Newport Green allows for the definition of cell subsets according to zinc content (18). TMRE selectively binds to mitochondrial membranes, allowing for detection of apoptosis (that results in decreased staining) (19-20). After washing, cells were stained with 7-aminoactinomycin D (7-AAD; Molecular Probes), which binds to DNA when cell membrane permeability is altered after cell death. Cell suspensions were analyzed (minimum 3.0×10⁴ events) using a FACScan cytometer (Becton Dickinson, Mountain View, Calif.) with the CellQuest software.

Human β-cell sorting: Dispersed cells were stained with NG and 7-AAD. Cell sorting was performed using a FACSvantage (Becton Dickinson). Cell subsets 7-AAD⁻, NG^{bright} and NG^{dim/negative} were collected separately (18). Control analysis showed that FACS sorting led to >90% purity.

Delivery of pro-apoptotic stimuli to islet cells: Islets were treated with selected compounds to induce apoptosis. The nitric oxide (NO) donor sodium nitroprusside (Baxter Healthcare Corporation, Deerfield, Ill.) was used at 0.5 mM for 18 hours. Hydrogen peroxide (H₂O₂; Sigma) was used at 200 μM for 18 hours. IL-1-β (50 U/ml) alone or in combination with TNF-α (1000 U/ml) and IFN-γ (1000 U/ml) was used for 24 hours. In additional experiments β-cell stress was induced by incubation of islet aliquots maintained as a pellet in a 15 ml conical tube for either 6 or 18 hours.

In vivo assessment of islet potency in the diabetic nude mouse model: Animal procedures were approved by the IACUC, and performed in the Preclinical Cell Processing Core. Male athymic nu/nu (nude) mice (Harlan Laboratories, Indianapolis, Ind.) were housed in Virus Antibody Free rooms in micro-isolated cages, having free access to autoclaved chow and water.

Animals were rendered diabetic via IV administration of 200 mg/kg of Streptozotocin (Sigma). Non-fasting blood glucose was assessed with a glucometer (Elite, Bayer; Tarrytown, N.Y.). Mice with sustained hyperglycemia (>300 mg/dl) were used as islet graft recipients.

2,000 human IEQ per recipient were transplanted under the kidney capsule (11,21-22) and non-fasting blood glucose values were assessed 3 times a week. Reversal of diabetes was defined as stable non-fasting blood glucose <200 mg/dl. Nephrectomy of the graft-bearing kidney was performed to confirm return to hyperglycemia and exclude residual function of the native pancreas in animals achieving normoglycemia after transplantation.

Statistical analysis: Data were analyzed using Excel for Windows software for descriptive statistics and data plotting. Data are shown as mean±standard deviation (SD). Statistical significance was considered for p values<0.05. Chi-square analysis was used to assess significant differences in the product of β-cell content (%) and viability (%) vs. transplantation success in immunodeficient mice. Logistic regression was then used to explore the influence of the two factors separately.

### Example 1

### Pre-Transplant Assessment of Fractional Beta Cell Viability Correlates with Clinical Islet Transplantation Outcomes

We have recently developed a method for the assessment of β-cell content and viability in HICP that allowed for >90% prediction of diabetes reversal in immunodeficient mice.

Methods: 29 transplanted HICP were assessed for β-cell content using immunofluorescence (iCys) and β-cell viability by FACS using a three-color analysis (7-AAD, Newport green, TMRE) allowing identification of dead and apoptotic cells, while distinguishing between β and non-β-cell subsets. Viable β-cell Equivalent Number/kg (vβEQ/kg) was calculated using the formula [islet β-cell content×β-cell fractional viability×IEQ/kg] and compared to clinical outcomes (i.e., insulin reduction rate and insulin independence) after islet infusion. Other calculations used the following formulae:

Viable β-cell index=(% β-cell content)×(% non-apoptotic β-cell)

β-cell Mass/kg==IEQ×(% β-cell content)/kg

Viable β-cell Mass/kg=IEQ×(% β-cell content)×(% non-apoptotic β-cell)/kg

Results: Average transplanted islet mass was 397,107±123,732 IEQ, 6,270±2,633 IEQ/kg, and 1,892±1,033 vβEQ/kg. β-cell content and β-cell fractional viability were 44±12% and 69±18%, respectively. Recipients of >8,000 IEQ/kg (n=7) achieved insulin independence (11,541±2,744 IEQ/kg and 4,138±1,583 vβEQ/kg). Amongst the recipients of <8,000 IEQ/kg (n=8), 3 became insulin independent (5699±407 IEQ/kg, 2,457±906 vβEQ/kg), while 5 didn't (6,335±1522 IEQ/kg; 1,085±458 vβEQ/kg).

Furthermore, analysis was performed dividing recipients according to insulin reduction rate (IRR)> or <60% after 1st islet infusion. Recipients of >8,000 IEQ/kg showed IRR>60% (9,983±2,450 IEQ/kg and 3,544±999 vβEQ/kg). Recipients of <8,000 IEQ/kg showed IRR of >60% (n=5; 4,825±1,325 IEQ/kg and 1,849±623 vβEQ/kg) and <60% (n=8; 4,827±1325 IEQ/kg and 983±385 vβEQ/kg).

Conclusions: Assessment of HICP by vβEQ/kg correlated with clinical outcome (P=0.027 and P<0.01 for insulin independence and IRR, respectively), while the amount of IEQ/kg transplanted was predictive only when very high number of islets were infused (>8,000 IEQ/kg). Our method for integrated assessment of HICP β-cell viability and content allows for prediction of post-transplants functional potency.

### Example 2

### A Method for the Assessment of Cellular Composition and Beta-Cell Viability in Human Islet Preparations

LSC allows precise definition of islet cell composition: As shown in FIG. 1C, meaningful data can be acquired by the use of marker-specific immunofluorescence analysis. FIG. 2A shows the correlation of β-cell percentages (LSC analysis) versus purity, the latter assessed by DTZ staining in 62 human islet preparations. The proportion of β-cells was much lower than expected based on DTZ staining, and quite widely disparate between preparations, even when ≧90% purity by DTZ staining was documented. We then calculated β-cell content in islets using the formula {[(β-cell %)×(β+α+δ+PP-cell %)⁻¹]×100}. The mean±SD of β, α, δ and PP-cells was 51.3±10.4, 36.0±9.4, 8.0±2.6 and 4.6±4.7, respectively (FIG. 2B). When analysis of cell composition was performed on preparations obtained from pancreata preserved in UW, and compared to preparations from pancreata preserved in PFC/UW, no significant differences were observed. These results indicate that analysis of islet purity by DTZ staining is largely inadequate, and that assessment of cellular composition by LSC represents a significant improvement toward objective evaluation.

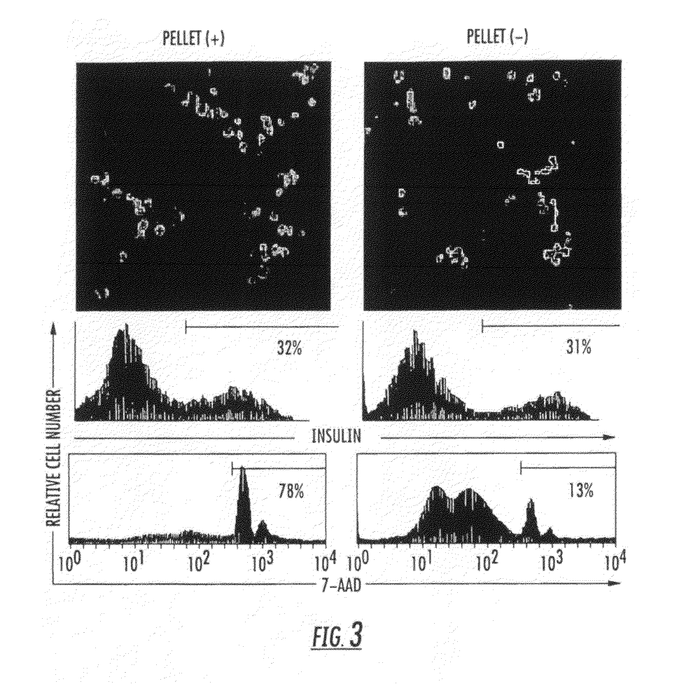

An important question was whether LSC analysis of endocrine cell subsets alone would allow for discriminating between live and dead cells. Experiments were performed where β-cell stress (resulting in death of a large percentage of cells) was induced by hypoxia/starvation. Islet cells maintained in pellet for 18 hours have little oxygen and nutrients available to support their viability and function. After the incubation period, dead cells were identified by 7-AAD staining. FIG. 3 shows that both live cells (control sample) and a sample containing mostly dead cells (78% 7-AAD⁺) were indistinguishable when stained with anti-insulin and anti-glucagon antibodies, indicating that analysis with LSC, while providing invaluable information on cellular composition, was not sufficient to estimate β-cell viability.

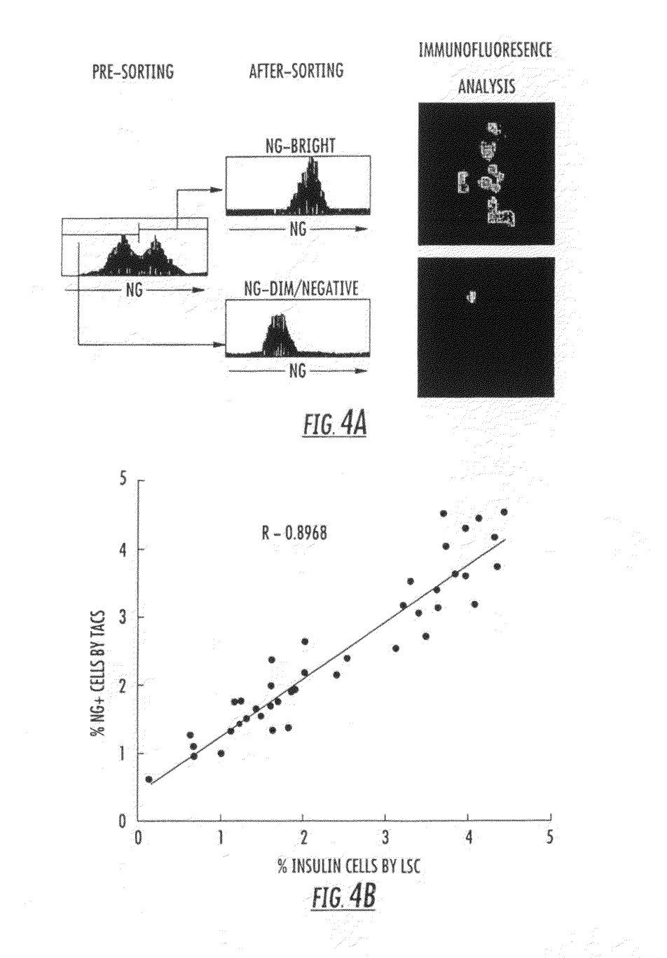

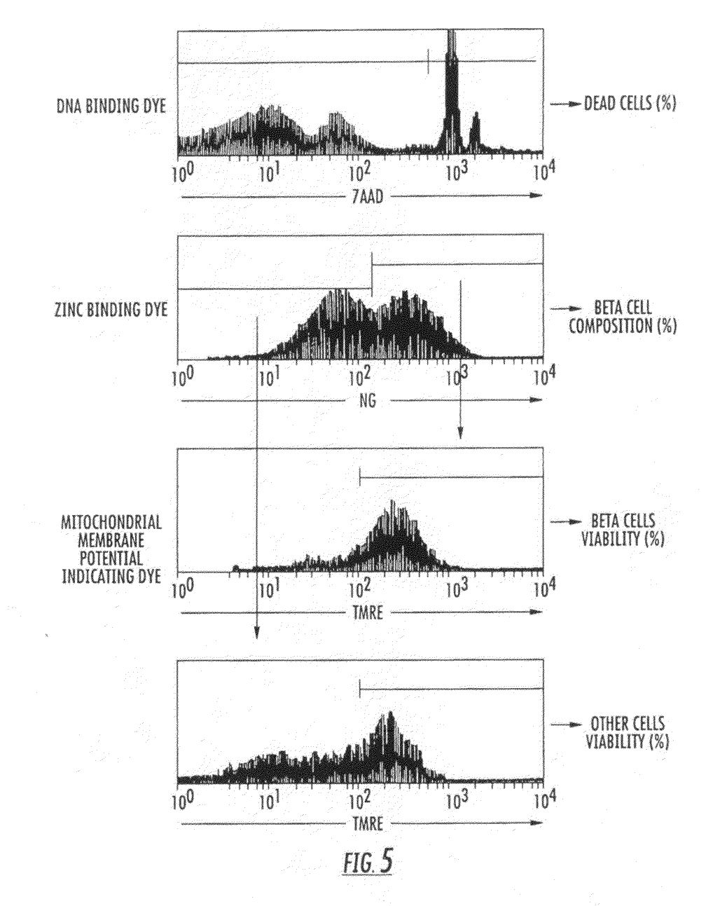

Newport Green staining allows identification of β-cells: We next utilized the zinc-binding fluorochrome NG to identify β-cells in virtue of their high zinc content. Single islet cell suspensions were incubated with both 7-AAD and NG and then analyzed by FACS. After exclusion of 7-AAD⁺ cells from further analysis, a bimodal pattern of NG staining was observed, with bright and dim/negative cells, as shown in FIG. 4A, upper left panel. Cells were then sorted into NG^{bright} and NG^{dim/negative} subsets (middle panels), and each subset was individually analyzed by immunofluorescence. Newport Green^{bright} cells appeared highly enriched for β-cells, while NG^{dim/negative} cells were highly depleted of β-cells (FIG. 4A, right panels), comprising other endocrine subsets and non-endocrine cells. Beta-cell identification was confirmed by functional analysis of in vitro glucose-stimulated insulin secretion: NG^{bright} cells responded, while NG^{dim/negative} cells had marginal insulin secretion.

The exclusion of 7-AAD⁺ (dead cells) from further analysis of NG staining could introduce a bias, excluding dead cells preferentially present in selected subsets. We therefore compared β-cell content of individual human islet preparations ascertained via FACS with that of LSC analysis. While the former excludes dead cells, the latter does not. There was a tight relationship between the results obtained with both techniques in all preparations tested (FIG. 4B), indicating that dead cells did not preferentially belong to any particular subset. Furthermore, our data on islet cellular composition are in keeping with data obtained by morphometric analysis performed on non-dissociated islets where endocrine cell subsets were evaluated after immunofluorescence staining and confocal imaging. In this set of experiments, β, α, and δ-cell percentages of islets from five randomly selected preparations were very similar to those obtained with LSC on dissociated cells from the same prep (Table 1).

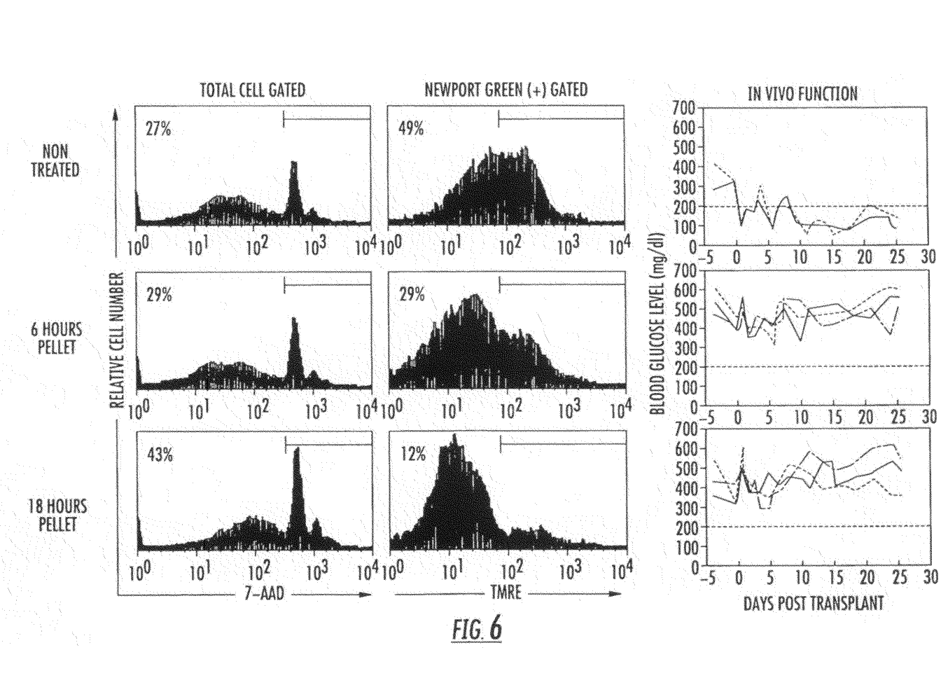

TMRE staining of NG-bright cells allows definition of viable and apoptotic β-cells: We evaluated apoptosis selectively in the NG^{bright} subset according to the method summarized in FIG. 5. After exclusion of dead cells from analysis by 7-AAD staining, live β-cells (NG^{bright}) were analyzed for mitochondrial membrane potential by staining with TMRE. In a complementary fashion, NG^{dim/negative} cells were also analyzed. This approach allowed for the definition of subsets of β-cells that differ in TMRE staining, discriminating between healthy and apoptotic β-cells (FIG. 6). Control islets were compared to aliquots incubated in hypoxic conditions for 6 or 18 hours. As expected, the proportion of TMRE⁻ β-cells increased as a function of time in hypoxic culture. However, the decreased TMRE staining in β-cells cultured in hypoxic condition for 6 hours was not paralleled by a change in the percentage of 7-AAD⁺ cells. This difference could be of critical importance to evaluate viability and potency of islet preparations. This is a further suggestion that conventional methods of islet cell viability assessment based on cell membrane integrity may be insufficient.

A clear correlation in fact was observed between TMRE staining and reversal of diabetes in transplanted immunodeficient rodents (FIG. 6) in preparations that were indistinguishable in terms of 7-AAD staining, indicating that analysis of viability with conventional methods could not always predict in vivo function: diabetes could not be reversed using islets cultured for 6 hours in hypoxic culture, where 7-AAD viability indicated a percentage of dead cells comparable to controls (29% vs. 27%, respectively). The additional analytical step with TMRE was necessary to predict functional outcome in the 6-hr pellet group (FIG. 6). A predictive value of DNA-binding dyes (7-AAD) could easily be observed in extreme hypoxic conditions (18-hr pellet).

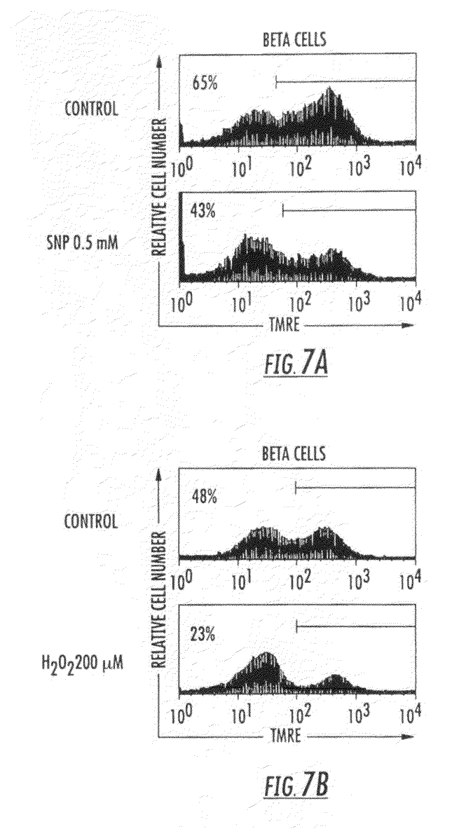

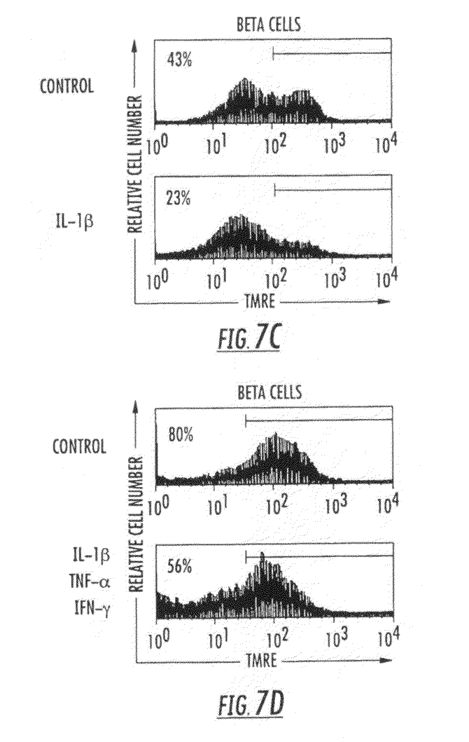

Analysis of β-cell viability/apoptosis reveals the noxious effects of pro-inflammatory stimuli. While the use of hypoxia/starvation is a quick and effective method to decrease islet cell viability and induce apoptosis, we wanted to confirm the value of our islet assessment method when other noxious condition were utilized. We evaluated the effects of reactive oxygen species (ROS), nitric oxide (NO) and cytokines, using the same method for analysis of β-cell apoptosis. All the selected noxious conditions utilized (SNP, H₂O₂, IL-1β and the cocktails of IL-1β, TNF-α and IFN-γ), led to an increase in the percentage of apoptotic β-cells (FIG. 7A), suggesting that our analytical method might be widely applicable.

Beta cell-specific analysis of viability/apoptosis allows precise quantification regardless of sample purity: Islet aliquots with different degrees of purity from 62 preparations were assessed with our method. In the representative sample of more than 100 independent experiments (FIG. 8A), TMRE⁺ cells in total living cells (7-AAD⁻ population) of preparations A, B and C were 61%, 67% and 50%, respectively (FIG. 8A, top 3 panels). These results were subsequently compared with β-cell-specific viability (FIG. 8A, bottom panels), where percentages of TMRE⁺ β-cells were 80%, 49%, and 82%, respectively. This remarkable discrepancy clearly shows the advantage of selectively analyzing β-cell viability by combining NG and TMRE staining. Analysis of NG^{bright} percentage (FIG. 8A, center panels) allows for the definition of β-cell mass in the preparation, and analysis of NG^{bright} TMRE⁺ percentages allows for the definition of the viable β-cell mass (FIG. 8A, bottom panels). Both parameters contribute significantly to the predictive value of our test. In more than 100 samples obtained from 62 human islet preparations with different purity, β-cells viability did not always correlate to other cells' viability and β-cell content.

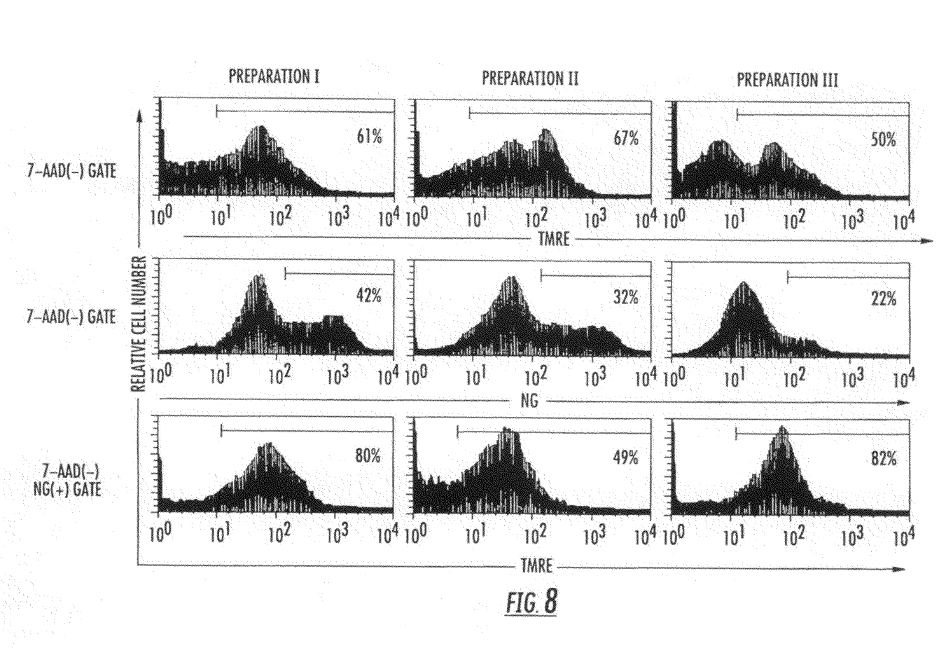

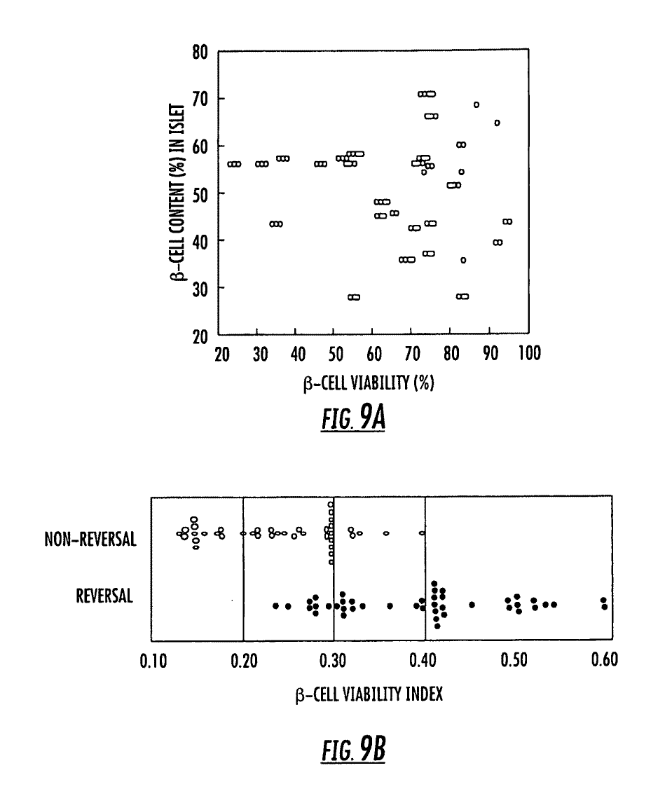

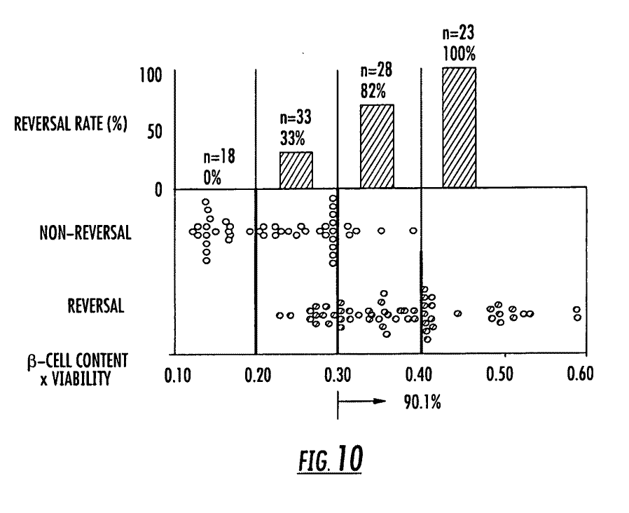

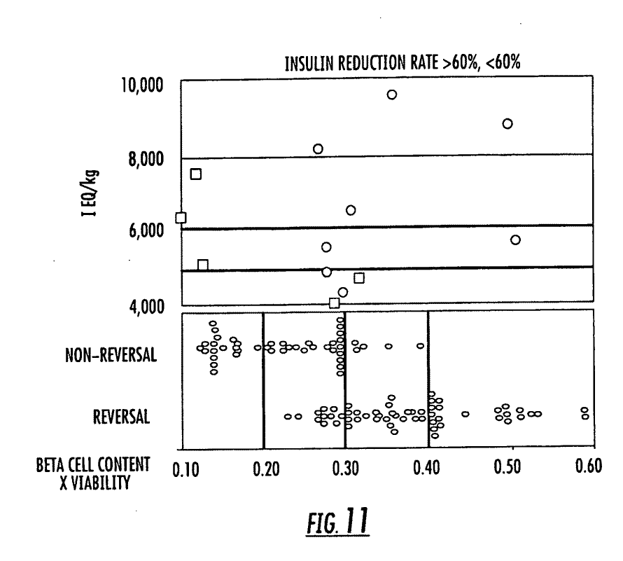

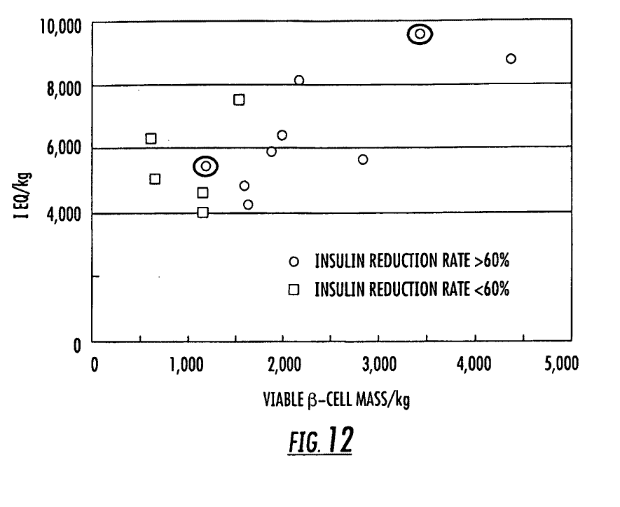

Furthermore, aliquots of 2,000 IEQ obtained from 24 individual islet preparations were transplanted to 82 diabetic immunodeficient mice. Beta-cell content (%) and β-cell fractional viability (%) were measured and plotted for each preparation, and related to transplant success or failure (FIG. 9A). This data analysis showed that success rate was higher when preparations had higher β-cell content and higher viability. The two values [β-cell content (%) and β-cell fractional viability (%)] were used to obtain a numeric product (β-cell viability index). This was then analyzed to seek a relationship with the in vivo assessment of islet potency (FIG. 9B). We show here that transplantation success rate has an evident relation with β-cell viability index. Chi-square analysis of β-cell viability index categories (<0.2, between 0.2 and 0.3, between 0.3 and 0.4, and >0.4) vs. success overwhelmingly suggests a positive association between index and success rate (p<0.0001); table 2 details the number of transplants and the success rates in the selected index categories. Logistic regression was then used to explore the two factors separately. When adjusting for β-cell content (%) in islets, β-cell fractional viability is an independent predictor that is significantly positively associated with success rate. The odds of success corresponding to a 10% increase in fractional viability is 3.78 (2.01, 7.08, p<0.0001) times greater for a fixed fraction of β-cells.

Discussion: We have demonstrated that a novel analytical method based on LSC and FACS analysis of islet preparations resulted in significant advantages, compared to the currently used techniques, providing objective information on islet cellular composition and islet cell subset viability (in particular β-cells).

LSC analysis of cellular composition appears as the most objective and sensitive means to determine the relative percentage of different cell subsets in islet preparations. More conventional techniques such as immunohistochemical (23-24) and electron microscopic (EM) analysis rely on subjective interpretation of the results and scoring based on observation of samples by the operator(s). The LSC hardware and software provide objective analysis of substantially higher number of cells (e.g. >10,000 cells) in a short time, as compared to the few hundreds that are generally counted manually or by operator-assisted imaging analysis.

Our results underline the inadequancy of DTZ staining to estimate β-cell mass, as preparations with similar purity assessed by DTZ actually contained significantly different β-cell masses (up to two-fold). While β-cell mass estimates are clearly a step forward in the definition of a dose-response relationship with transplantation outcome, the data presented here suggests that analysis of β-cell viability by TMRE is of critical value for prediction of transplant outcome. Our results clearly indicate that there is a correlation between viable β-cell mass and transplantation outcome in immunodeficient rodents, which is considered to date the most reliable in vivo biologic test to assess potency of an islet preparation. Our finding that DNA-binding dye exclusion does not always correlate with graft outcome is another strong argument to suggest the inadequacy of this test, when performed as the only assay of islet viability.

We also show that our method to evaluate β-cell viability is capable of detecting damage mediated by different noxious conditions, including ischemia/hypoxia, H₂O₂, NO, IL-1β and cytokine cocktails (25-27). This suggests that the method is sensitive enough to be of assistance in the detection of islet cell damage possibly resulting from different conditions related to donor brain death, pancreas procurement and preservation, as well as islet processing.

Our choice of the reagents used in the FACS three color analysis was based on their full compatibility in terms of wave length emission, so that each can be easily descriminated, while providing the needed information. Furthermore, the selection of NG was based on recently reported high specificity and sensitivity for β-cells (18), and extensive literature review indicated that TMRE could be a suitable marker for the study of mitochondrial membrane potential (19-20). Our data argues in favour of the efficacy of this novel method in selectively detecting β-cell viability, and suggests that a correlation exists between β-cell fractional viability and in vivo function.

Additional methods to predict islet function have been proposed, including analysis of oxygen consumption (28) and analysis of ATP levels (29,30). While the theoretical premises to utilize these methods are certainly strong, we feel that there could be difficulties in using them as single, straight-forward analytical tests of islet potency. The major problem that characterizes both tests is that the read-outs do not allow for the definition of β-cell contribution to the overall result, since all other endocrine cells within the islets and contaminant non-endocrine cells present in the preparation contribute to oxygen consumption and ATP levels. For example, in islets with a purity of 50% (50% non-endocrine tissue) and a β-cell composition of 35% (35% of endocrine cells are β-cells), the contribution of β-cells to oxygen consumption or ATP levels could be approximately 18% of the total. If β-cells are 100% or 30% viable, the relative change in oxygen consumption could be approximately 13% of the total. Our data also suggests that assessment of the overall viability in islet cells poorly correlates with β-cells viability, and rather leads to either under- or over-estimation of β-cells fractional viability. Nevertheless, integration of cellular composition assessment to discriminate the contributions of β- and non-β-cells with methods able to determine oxygen consumption or ATP contents will represent a valuable strategy to improve our analytical analysis in the near future.

Our data on cellular composition are in keeping with data obtained with morphometric analysis of sections of isolated human islets where endocrine cell subsets were evaluated after immunofluorescence staining by confocal imaging. In this set of experiments, β, α and δ cell percentages were very similar to those obtained with LSC on the same preparations after dissociation.

Advantages of our method include the fact that we can determine β-cell mass and viability in a very short time, in a fully objective manner, on large size samples (≧30,000 cells), with little expense, and by the use of an instrument (flow cytometer) that is readily available to most facilities that isolate islets for research and transplantation.

In summary, we report a novel method for the assessment of cellular composition and β-cell viability in human islet preparations. This method for islet assessment would be of importance in the identification of pre-transplant product release criteria that are predictive of post-transplant outcome, as recommended by the Food and Drug Administration before a biologic license for islet cell products could be considered (14,15).

### Example 3

### Refining the Assessment of Human Islet Cell Products

The evaluation of specific cell subset viability (PDC and β-cells) is performed simultaneously as well as for the detailed phenotypic analysis of PDC and acinar cells.

Human islet isolation and purification: Human islet isolations were performed from pancreata preserved with University of Wisconsin solution alone or with pre-oxygenated two-layer perfluorocarbon (27) at the Human Cell Processing Facility of the University of Miami School of Medicine. Islets were isolated using a modified automated method (Ricordi C, et al. Diabetes 1988; 37: 413-420).

Islets were purified using a computerized semi-automated cell processor (Cobe 2991; COBE Laboratories, Inc. Lakewood, Colo.) in a refrigerated (4° C.) cell processing room. The standard procedure for the purification of human islet preparation consisted of a continuous gradient purification performed by loading 1.100 g/mL (osmolality 320-373 mOsm/kg/H₂O) and 1.077 g/mL (285-327 mOsm/kg/H₂O). Ficoll-based density gradients in the doughnut-shaped bag using a gradient maker device, followed by top-loading of the pancreatic digest (≦20 mL of tissue/run) in UW solution (29). After 5 min of centrifugation at 2,400 rpm, approximately 15 fractions were collected, and examined for purity. Islet fractions were combined based on the purity assessed by DTZ. In general, the fractions with highest islet purity were collected from the low-density fractions 4-7 (density 1.081-1.085 g/ml). Medium and high-density islet fractions were combined with fractions 7-10 and 11-14 (1.86-1.090 and 1.091-1.095 g/ml, respectively).

Islet cell dissociation: Single cell suspensions were obtained by incubating aliquots of approximately 1,500-2,000 islet equivalents (IEQ) in 1 ml Accutase (Innovative Cell Technologies, Inc, San Diego, Calif.) at 37° C. for 10-15 min, as described (Ichii H, et al. Am J Transplant. 2005; 5(7):1635-1645).

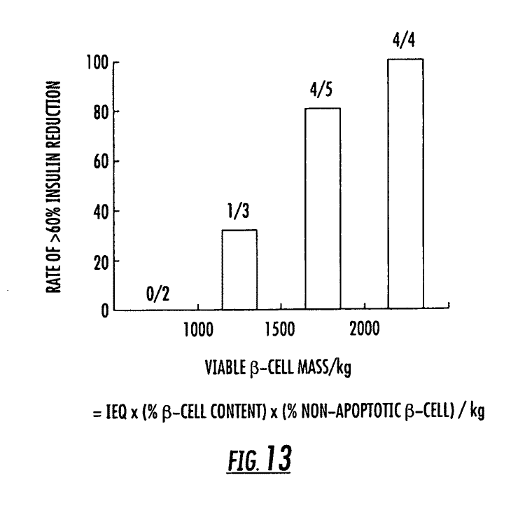

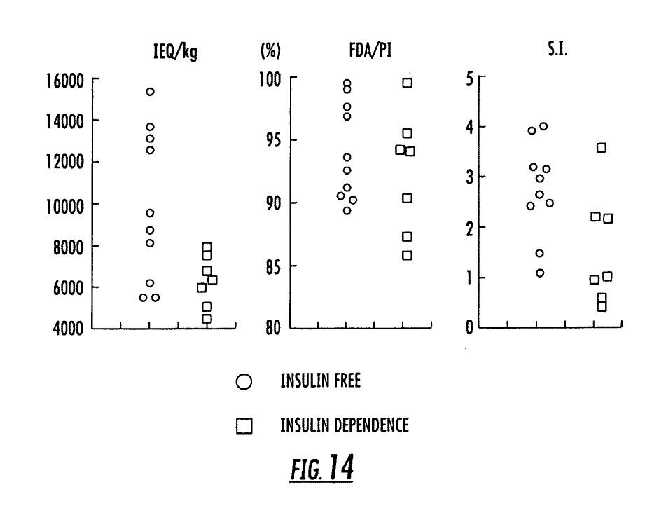

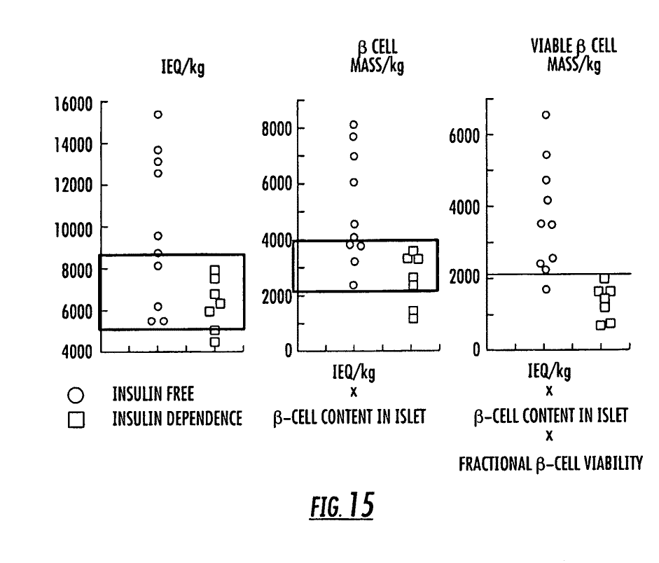

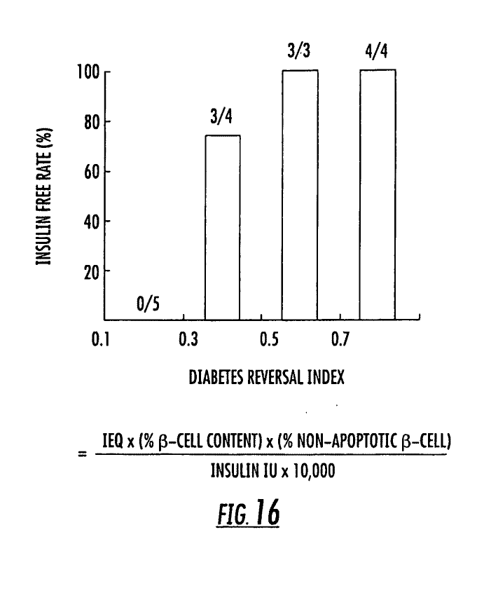

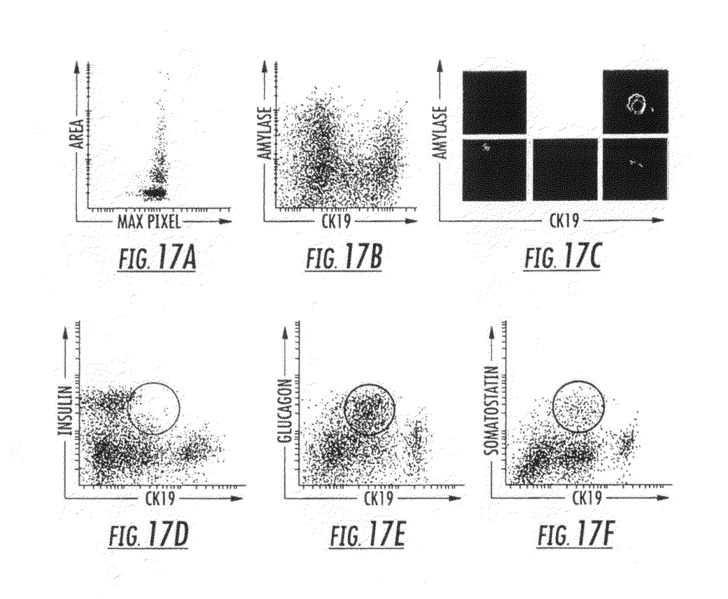

Assessment of PDC content using LSC/iCys: Dispersed cells from islet preparations were fixed on glass slides with 2.5% paraformaldehyde (Electron Microscopy Sciences, Washington, Pa.). After incubating with Protein Block (Bio-Genex, San Ramon, Calif.) and washing (Optimax Wash Buffer; Bio-Genex, San Ramon, Calif.), cells underwent 1-hour incubation with primary antibodies: mouse monoclonal anti-cytokeratin (CK)-19 (1:50; Protein Tech Group, Inc, Chicago, Ill.); rabbit polyclonal anti-CK19 (1:200; Dako North America, Inc, Carpinteria, Calif.); mouse monoclonal anti-CA19-9 (1:100; Novocastra, UK) and rabbit polyclonal anti-amylase (Amy; Dako). Secondary antibodies included Alexa Fluor-488 goat anti-mouse IgG or Alexa Fluor-647 goat anti-rabbit IgG (1:200; Molecular Probes, Eugene, Oreg.). Nuclear staining was obtained with 4′,6-diamidino-2-phenylindole (DAPI; 1:300; Molecular Probes). Samples were analyzed using a Laser scanning cytometer (LSC) or an iCys (CompuCyte, Cambridge, Mass.). Slides were scanned at 40×. Each fluorescent positive event (selective marker specific for the target cell population) was recorded using the argon/HeNe/Violet laser. Single cells were identified and gated according to the DAPI staining area (FIG. 17A). Cells from every subpopulation were visualized directly in the LSC/iCys by re-localization to confirm regular morphology (FIG. 17C). A minimum of 5,000 cells was acquired and analyzed for each sample (Ichii H, et al. Am J Transplant. 2005; 5(7):1635-1645).

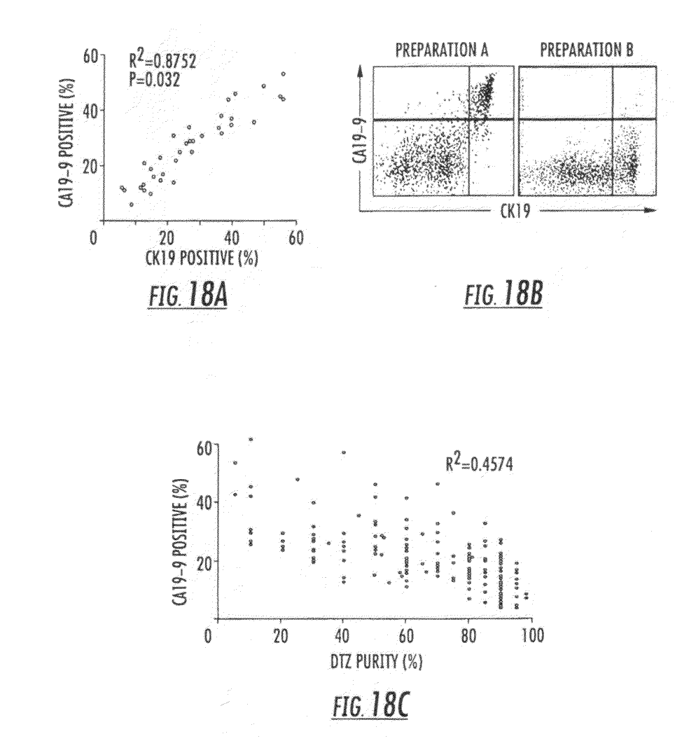

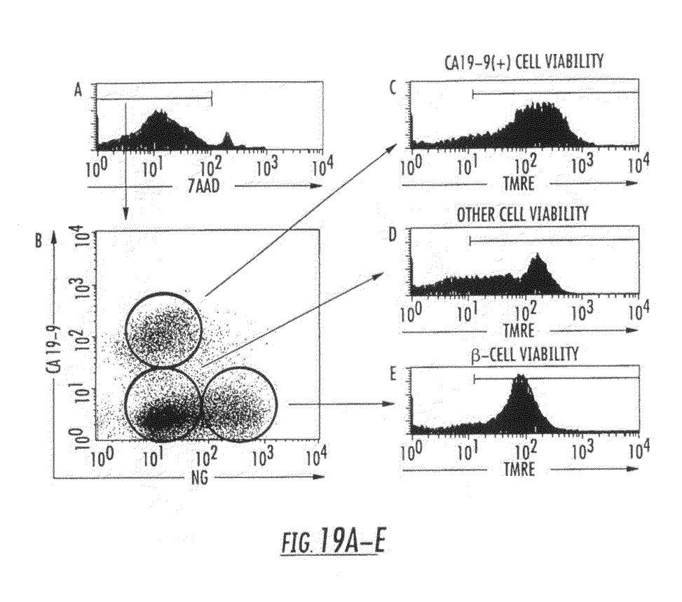

Assessment of PDC-specific viability: Single cell suspensions were incubated for 30 min at 37° C. in phosphate buffered saline (PBS) without Ca²⁺ and Mg²⁺ with Newport Green PDX acetoxymethylether (NG; 1μ) and tetramethylrhodamineethylester (TMRE; 100 ng/ml; both from Molecular Probes). Cells were incubated with anti-CA19-9 antibody (1:100) for 15 min followed by goat anti-mouse antibody (Alexa-647). After washing, cells were stained with 7-aminoactinomycin D (7-AAD; Molecular Probes), a marker of cell death. Cell suspensions were analyzed (minimum 3.0 104 events) using a FACScan cytometer with the CellQuest-pro software (Becton Dickinson, Mountain View, Calif.). We have evaluated apoptosis selectively in the NG^{bright} and CA19-9+ cells according to the scheme in FIGS. 19A-E. After counting, dead cells (7-AAD+) were excluded from further analysis, live β-cells (NG^{bright}) and PDC (CA19-9⁺) were analyzed for mitochondrial membrane potential (TMRE), which allows discriminating between healthy (TMRE⁺) and apoptotic (TMRE⁻) cells.

Delivery of pro-apoptotic stimuli to islet and non-endocrine cells: Islets were treated with selected compounds for 24 hours to induce apoptosis, as described (Ichii H, et al. Am J Transplant. 2005; 5(7):1635-1645), including the nitric oxide donor, S-Nitroso-N-acetyl-DL-penicillamine (SNAP; 0.5 mM, Sigma); hydrogen peroxide (H₂O₂ 200 μM; Sigma); or cytokine cocktail with IL-1-β (50 U/ml), TNF-α (1,000 U/ml) and IFN-γ (1,000 U/ml). In addition, hypoxic stress was induced by a 6-hour incubation of islet aliquots as a pellet into a 15 ml conical tube filled with culture medium (Ichii H, et al. Am J Transplant. 2005; 5(7):1635-1645; Ichii H, et al. Am J Transplant. 2007;7(4):1010-1020).

Human PDC sorting: Dispersed cells were incubated with anti-CA19-9 antibody for 30 minutes followed by a 20-min incubation with magnetic beads coated with a anti-mouse IgG (Miltenyi Biotec, Auburn, Calif.). The cell suspension was passed through a magnetic column to obtain positive selection of CA19-9⁺ cells. The efficiency of sorting was confirmed by FACS analysis (Gmyr V, et al. Biochem Biophys Res Commun. Jul. 16, 2004;320(1):27-33).

Production of inflammatory mediators by PDC: During islet purification by density gradients, pancreatic tissue fractions with different densities (low=1.080-1.085, medium=1.086-1.090 and high=1.091-1.095 g/ml) were collected. Human PDC were sorted from high and low density fractions obtained from five independent islet preparations. Enriched PDC (1×10⁶/ml) were cultured in Miami-defined medium (Mediatech) for 24 hours (Ichii H, et al. Am J Transplant. 2007;7(4):1010-1020). Concentrations of pro-inflammatory mediators (namely, IL-1β, IL-6, IL-8, IFN-γ, MCP-1, MIP-1β, and TNF-α) in islet supernatants were determined using Multi-Plex cytokine kits following the manufacturer's protocol (Bio-Plex; Bio-Rad Laboratories). Additionally, PDC were homogenized, and TF measured by ELISA (Imubind Tissue Factor, American Diagnostica, Greenwich, Conn., USA). The amount of cytokines, chemokines and TF was normalized by total PDC protein content.

Statistical analysis: Data were analyzed using Excel for Windows software for descriptive statistics and data plotting. Data are shown as mean±standard deviation (SD). Statistical significance was considered for p-values<0.05. Logistic regression was then used to explore the influence of the two factors separately.

### Results

Phenotypic analysis of non-endocrine cell in human islet preparations using LSC/iCys: To assess the proportion of non-endocrine cells in islet preparations, amylase (Amy) and CK19 were used for the identification of acinar cells and PDC, respectively. This led to the identification of five cell subpopulations (FIG. 17B). Although LSC/iCys analysis clearly showed Amy⁺CK19⁻ cells indicating exocrine cells, another cell subset Amy⁺CK19^{bright} was also identified. Furthermore, an Amy⁻CK19^{dull} cell population was observed. In order to characterize this Amy⁻CK19^{dull} population, immunostaining for insulin, glucagon and somatostatin was combined with CK19. The LSC/iCys analysis revealed that α-cells and δ-cells, but not β-cells, do express CK19dull (FIG. 17D-17F). These results indicated that single staining for CK19 is not specific for evaluating PDC content in human islet preparations, and that LSC/iCys analysis allows for detailed phenotypic analysis of endocrine and non-endocrine cell subsets.

CK19 is an intracellular protein and therefore fixation of cells is necessary for its detection by immunostaining precluding its application on live cells. The carbohydrate antigen 19-9 (CA19-9) has been described as a pan-ductal membrane antibody for human PDC. Therefore, we evaluated the expression of CK19^{bright} cells in 34 human islet preparations and compared it to that of CA19-9 using LSC/iCys. A positive correlation between CK19^{bright} and CA19-9+ expression was observed in human islet preparations (R2=0.8752, p=0.032)(FIG. 18A). Moreover, α-cells and δ-cells expressing CK19^{dull} resulted negative to CA19-9, while a fraction of CA19-9⁺ cells expressed Amy. Interestingly, 5.0% of the human islet preparations tested ( 8/161) expressed CK19 but not CA19-9 (FIG. 18B).

We examined 193 purified fractions from 10⁶ human islet preparations with different purities assessed by Ditizone (DTZ) and compared to PDC content by CA19-9 immunostaining. The proportion of CA19-9⁺ cells in islet preparations was higher than expected even when purity>90% by DTZ were estimated (i.e., 3-30% CA19-9⁺; FIG. 18C). These results indicate that PDC analysis by CA19-9 antibody using FACS represents a simple, quick and objective method for the evaluation of PDC content in human islet preparations.

Analysis of PDC viability by FACS using CA19-9 and TMRE staining: We have developed an analytical method to assess simultaneously the viability of PDC and β-cells by FACS (FIGS. 19A-19C). As in our previous study (26), viable (non-apoptotic) β-cells and PDC were analyzed for mitochondrial membrane potential by TMRE. In a complementary fashion, NG^{dim/negative} and CA19-9− cells were also analyzed.

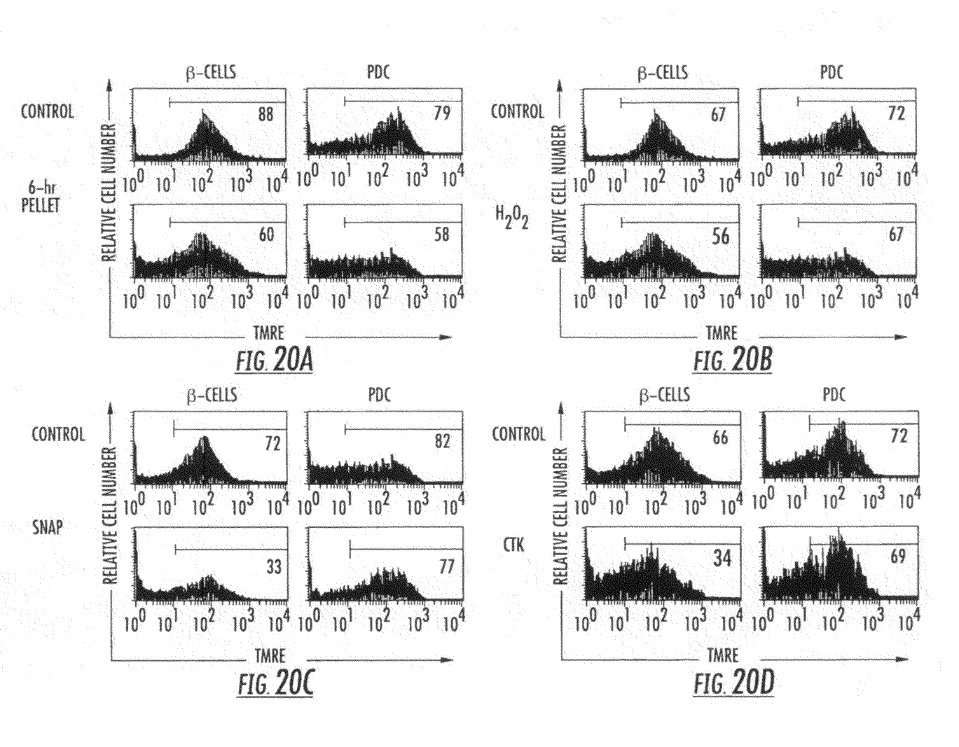

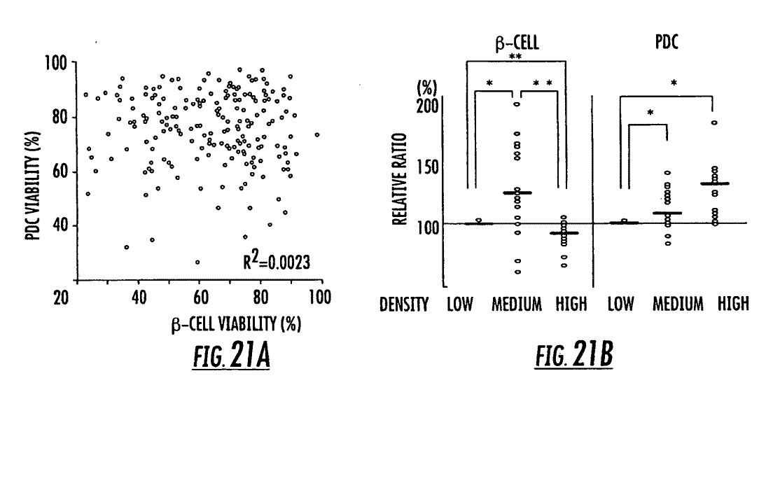

To confirm the value of our refined PDC and β-cell viability assessment method, we evaluated the effects of hypoxic/starving conditions (FIG. 20A), reactive oxygen species (ROS)(FIG. 20B), nitric oxide (NO)(FIG. 20C) and cytokine cocktail (IL-1β, TNF-α and IFN-γ)(FIG. 4D). All these noxious conditions tested led to a measurable decrease in the proportion of viable PDC and β-cells (FIGS. 20A-20C). Moreover, we observed that PDC are more resistant than β-cells to noxious conditions (FIGS. 20A-20C).

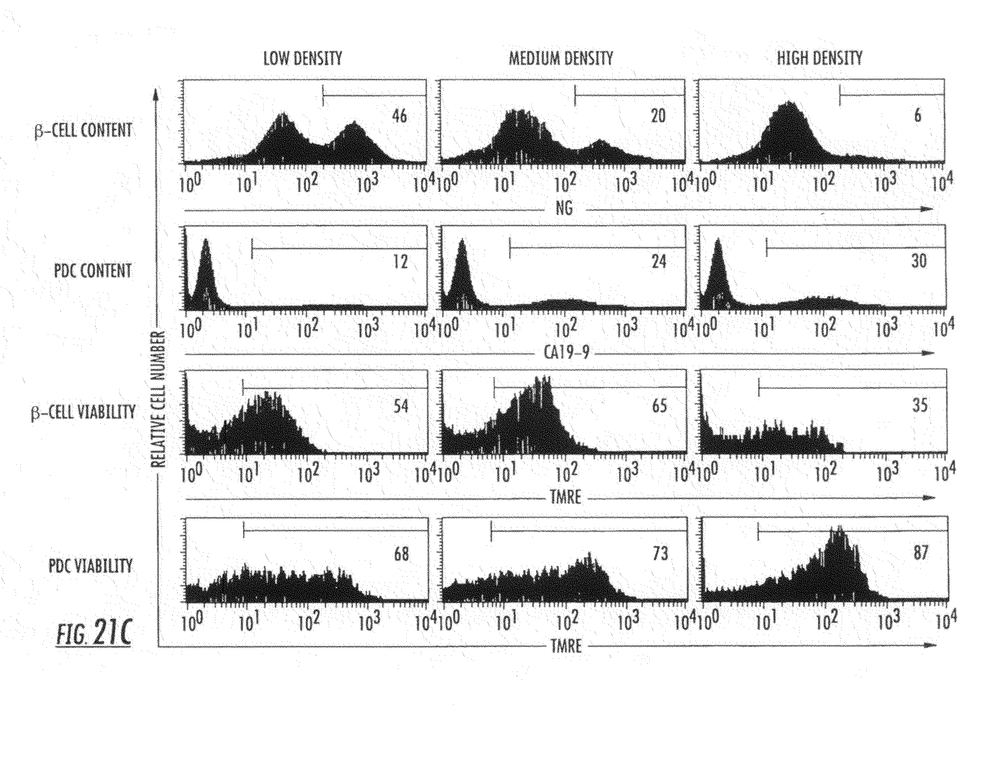

Analysis of PDC-specific viability in islet preparations with different degrees of purity: We have examined PDC and β-cell viability in 202 fractions from 124 human islet preparations. The average of PDC viability was higher than that of β-cells (PDC vs. β-cell: 75.5±13.9% and 62.7±18.7%, respectively; p<0.0001). There was no correlation between PDC and β-cell viability (R2=0.0023)(FIG. 21A).

We selected 31 human islet preparations from which purity fractions from three different density layers (low=1.080-1.085, medium=1.086-1.090 and high=1.091-1.095 g/ml) were available for analysis. The islet purity for each density fraction ranges: 95-71% (high), 70-4 (medium) and 40-10% (low), respectively. PDC and β-cell viability in each fraction was analyzed by FACS. The relative ratio of PDC and β-cell viability within medium and high density fractions was calculated in regards to that obtained in the low density fraction. The highest PDC viability was observed in fractions obtained from high density, and decreased in those with lower density (medium/low=1.15±0.33, p<0.05; high/low=1.31±0.53, p<0.05)(FIG. 21B). However, the analysis of β-cells showed that the relative viability in medium and low density fractions was significantly higher and lower, respectively, when compared to that of low density fraction (middle/low=1.24±0.35, p<0.005; high/low=0.91±0.10, p<0.005)(FIG. 21B).

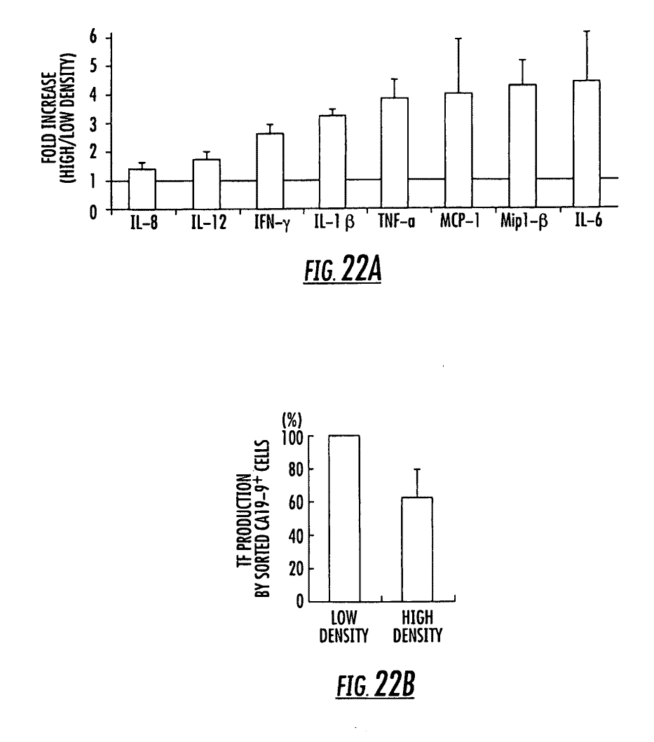

Cytokines, chemokines and Tissue Factor production by CA19-9+ cells: To evaluate the function of PDC from different density fractions after islet purification, we sorted PDC using CA19-9 antibody. Sorted PDC were incubated in culture medium for 24 hours and supernatant collected for the measurement of inflammatory mediators. Recovered cells were homogenized for the measurement of TF, which has been shown to be relevant to clinical outcomes (Moberg L, et al. Lancet. 2002; 360(9350):2039-2045; Johansson H, et al. Diabetes. 2005;54(6):1755-1762). Collectively, cytokine and chemokine production from PDC in isolated from heavy density fractions was higher, when compared to that of low density fractions, suggesting that PDC with higher viability are able to produce larger amounts of proinflammatory mediators (FIG. 22A). On the other hand, a significant reduction in TF production was observed from PDC obtained from high density fractions, when compared to that of low density (FIG. 22B-22C). These results indicate that the proportion and viability of PDC in human islet preparations might be considered as one of the important factors that could affect islet graft function.

Discussion: The development of reliable potency tests is one of critical requirements in the field of islet transplantation. The presence of significant amounts of non-endocrine cells within the final islet cell product makes it difficult to assess the β-cell specific viability. In the present study, we demonstrated that a novel analytical method based on the use of three different dyes (namely, 7-AAD, NG and TMRE) and one surface (anti-CA19-9) antibody provides precise and objective information on the viability and content of PDC as well as of β-cells. Furthermore, the possibility of performing the assay on both β-cells and PDC simultaneously on a single sample obtained from the final cellular product regardless of the purity represents a significant advantage, when compared to the currently used techniques.

The outcomes of clinical islet transplantation have been greatly developed owing to the introduction of more efficient methods for the separation of islets and more effective immunosuppressive strategies. However, almost 90% of the recipients of allogeneic islets in recent trials required reintroduction of insulin within 5 years (Shapiro A M, et al. N Engl J Med. 2006; 355(13):1318-1330; Ryan E A, et al. Diabetes. 2005; 54(7):2060-2069). This currently limited success of islet transplantation imposes us to assess possible reasons for such an outcome, and try to make changes that can allow for long-term islet graft function. Multiple factors may have contributed to the limited function of transplanted islets in the clinical setting. It is conceivable that a reduction of islet cell regeneration potential in the recipients may have occurred probably due to the adverse effect of immunosuppressive drugs, the place of implant (liver) may be unsuitable for islet regeneration, or/and the loss of β-cell precursor in islet preparations.

Insulin-secreting cells have been obtained in vitro from diverse sources, including embryonic stem cells. Several studies suggest that somatic stem cells can give rise to insulin-producing cells, including hepatic oval cells, splenic-derived cells, pancreatic nestin-positive cells and marrow-derived cells. PDC are closely associated with β-cells in the human pancreas and have been shown to give rise to endocrine islet cells in both rodents and humans. Beneficial effects of PDC on islet cell viability have been described, which may be partially attributed to their ability of producing IGF-II. On the other hand, PDC may have detrimental effects on islet engraftment and survival. Exposure of PDC to interleukin-1β (IL-1β) and interferon-γ (IFN-γ) can result in nitric oxide production in human islets infiltrated by cytokine-releasing immune cells. Moreover, contaminating PDC may contribute to early β-cell damage after intrahepatic islet transplantation through their expression of TF that may exert a potent factor VII-dependent pro-coagulant activity. Immunogenicity of PDC may also relate to the expression of CD40, a member of the TNF-receptor family that was initially described on B cells, activated monocytes, dendritic, endothelial and epithelial cells as well as fibroblasts. In addition, we recently found that purified pancreatic β-cells express a functional CD40 and that, following engagement with its ligand (CD154) results in the secretion of proinflammatory mediators including IL-6, IL-8, monocyte chemoattractant protein-1 (MCP-1) and macrophage inflammatory protein (MIP-1β) (Klein D, et al. Diabetologia. 2005; 48(2):268-276; Barbe-Tuana F M, et al. Diabetes. 2006;55(9):2437-2445). The CD40-CD154 co-stimulation pathway plays a pivotal role in numerous T-cell-mediated inflammatory disorders.

Human islet grafts include a sizable mass of non-endocrine tissue that is transplanted along with endocrine cells and that may contribute to the early and long-term outcome. Rodent studies indicate that the composition of islet grafts, both with respect to endocrine and non-endocrine cell subsets, may influence long-term metabolic function. Moreover, a positive correlation between the number of CK19⁺ cells and the long-term metabolic success of clinical islet transplantation has been recently proposed, a phenomenon that may result from a greater frequency of islet cell neogenesis from putative PDC precursors.

Prior to the present study, there was no assessment method for the detailed characterization of PDC content and viability in human islet preparations. Analysis of non-endocrine cells through LSC/iCys revealed that a substantial amount of Amy⁺ cells also expressed CK 19, suggesting that a single staining with CK19 could lead to an overestimation of PDC numbers by counting also acinar cell subsets. Moreover, it appears difficult by the means of conventional immunohistochemical techniques distinguishing CK19^{high} from CK19^{dull} cells, which are mainly composed of α-cells and δ-cells. Conversely, the analysis performed by the means of LSC/iCys allowed for an objective and detailed phenotypic analysis of higher numbers of cells in a relatively short time, when compared to the few hundreds that are generally counted manually or by operator-assisted imaging analysis. Moreover, PDC viability might represent a critical variable for both inflammation and regenerative potential after transplantation. Our data indicates that there was wide variability in terms of PDC viability based on the analysis of more than 200 human islet fractions. Interestingly, we observed that PDC with higher viability were obtained from higher density purification fractions (lower purity). In addition, higher cytokine and chemokine levels were obtained from more viable PDC. The combination of accurate PDC content and viability assessment in islet preparations may provide a more accurate prediction of short- and long-term graft function. It is conceivable that if PDC regenerative potential may have a positive impact on long-term islet graft function, both qualities (namely, viability) along with numbers of PDC composing the graft should matter. Therefore, our assessment method for islet cell products to include evaluation of PDC quality and quantity may be of assistance in better determining the contribution of non-endocrine cells to engraftment and long-term function in clinical islet transplantation and possibly also to perform precise phenotypic cell analysis relevant for pancreatic stem cell research.

The pan-ductal cell marker CA19-9 is expressed on the surface of human PDC by reacting with sialosyl-fucosyl-lacto-tetraose, corresponding to sialylated blood group antigen Lewis A that is a product of the Le gene (34). Although CA19-9 could provide useful information on PDC content in human islet preparations, 5% ( 8/161) of cell products lacked of CA19.9⁺ cells even if a substantial amount of CK19⁺ cells was present in the samples. Interestingly, the colon tissue of the Japanese patients possessing the Le allele that encodes the active Lewis enzyme either homozygously (Le/Le) or heterozygously (Le/le) expressed CA19-9, and that the 5% of the patients were le/le homozygotes and therefore lacked CA19-9 expression. In light of this observation, CK19 should be also used for the evaluation of PDC content in order to confirm CA19-9 expression.

The need for reliable predictive islet cell product tests has prompted the development of multiple potency assays such as oxygen consumption rate, analysis of ATP levels, and the measurement of reactive oxygen species. All proposed methods have shown promise regarding their predictive value on graft function into chemically-diabetic, immunodeficient mice receiving high purity human or porcine islets (the latter generally ˜80-90% β-cell content). However, clinical islet preparations are composed not only of β-cells, but also by a substantial amount of non-endocrine and endocrine (α- and δ-cells) cellular subsets. The most difficult part in islet potency testing is to identify β-cell-specific contribution to the results obtained in each of these test. Notably, for potency assays that do not discriminate for cell subsets composing the sample, only if each cell subset (endocrine and not) within the same islet preparation has similar viability the result of the test may be representative to β-cell viability. According to the analysis of more than 150 islet preparations in our study, the viability and content of PDC and of β-cells wide varied and there was no correlation between PDC and β-cell values. These results clearly indicate that the assessment of heterogeneous cell populations such as ADP/ATP ratio and FDA/PI cannot represent β-cell-specific viability.

Another critical issue to be addressed in islet potency testing is whether the viability of islet aliquots obtained from the purest layers can safely represent that of an entire final islet preparation with lower purity. Our data showed that β-cell viability in medium and high-density fractions was significantly higher and lower respectively, when compared to that of the low-density fraction. In addition, high-density fractions showed the highest PDC viability, which decreased linearly in lower density fractions. These results indicates that the viability assessment of the purest islet fractions may not reflect adequately that of PDC or β-cells in whole islet preparations, and that the sample for islet quality assessment and β-cell viability should be obtained from a mixed fraction of the final islet cell product after mixing pure and impure fractions prior to implantation. Therefore, some of islet quality assessment tests evaluating whole islet cells (i.e., FDA/PI and ADP/ATP ratio) may overestimate β-cell viability due to the contribution to the readout of PDC with higher viability composing the final preparation.

While it is apparent that islet cell quality is critical for clinical islet transplantation outcome, other factors such as inflammation mediators should be taken into account for overall assessment of islet cell products. Indeed, MCP-1 secreted by islet preparations may negatively affect the clinical outcome of islet transplantation. Additionally, islet-produced TF produced by islet preparations may trigger detrimental thrombotic reactions at the time of islet infusion. PDC could produce pro-inflammatory cytokines and chemokines as well as TF that may have detrimental effects on islet cell viability and function, leading to impaired islet engraftment. In our study, sorted PDC from high-density fractions could produce higher amount of pro-inflammatory mediators, when compared to PDC from low-density fractions. However, the production of TF from low-density fractions was higher than that of low-density fraction. These results suggest that PDC viability and content in islet preparation might be critical variables contributing to clinical outcome of islet grafts.

In summary, we have established a novel method for the simultaneous assessment of PDC and β-cell content and viability in human islet preparations. Our data suggests that PDC contaminating islet preparations may influence clinical outcomes after transplantation. Future efforts will focus on the better understanding and optimization of methods for isolation and culture of PDC for transplantation. The precise assessment of PDC content and viability along with that of β-cells in human islet cell products may be of assistance in developing strategies to enhance islet engraftment and long-term function.

### REFERENCES

1. Ricordi C, Strom T B: Clinical islet transplantation: advances and immunological challenges. Nat Rev Immunol. 2004; 4(4):259-268.
2. Shapiro A M, Lakey J R, Ryan E A, Korbutt G S, Toth E, Warnock G L et al. Islet transplantation in seven patients with type 1 diabetes mellitus using a glucocorticoid-free immunosuppressive regimen. N Engl J Med. 2000; 343(4):230-238.
3. Markmann J F, Deng S, Huang X, Desai N M, Velidedeoglu E H, Lui C et al. Insulin independence following isolated islet transplantation and single islet infusions. Ann Surg. 2003; 237(6):741-749.
4. Hering B J, Kandaswamy R, Harmon J V, Ansite J D, Clemmings S M, Sakai T et al. Transplantation of cultured islets from two-layer preserved pancreases in type 1 diabetes with anti-CD3 antibody. Am J Transplant. 2004; 4(3):390-401.
5. Goss J A, Goodpastor S E, Brunicardi F C, Barth M H, Soltes G D, Garber A J et al. Achievement of insulin independence in three consecutive type-1 diabetic patients via pancreatic islet transplantation using islets isolated at a remote islet isolation center. Transplantation. 2002; 74(12):1761-1766.
6. Ault A: Edmonton's islet success tough to duplicate elsewhere. Lancet. 2003; 361(9374):2054.
7. Shapiro A M, Ricordi C, Hering B: Edmonton's islet success has indeed been replicated elsewhere. Lancet. 2003; 362(9391):1242.
8. Ricordi C: Human islet cell transplantation: new perspectives for an old challenge. Diabetes. 1996; review 4:356-369.
9. London N J, Contractor H, Lake S P, Aucott G C, Bell P R, James R F: A microfluorometric viability assay for isolated human and rat islets of Langerhans. Diabetes Res. 1989; 12(3):141-149.
10. Latif Z A, Noel J, Alejandro R: A simple method of staining fresh and cultured islets. Transplantation. 1988; 45(4):827-830.
11. Ricordi C, Gray D W, Hering B J, Kaufman D B, Warnock G L, Kneteman N M et al. Islet isolation assessment in man and large animals. Acta Diabetol Lat. 1990; 27(3):185-195.
12. Berney T, Pileggi A, Molano R D, Ricordi C: Epithelial Cell Culture: Pancreatic Islets. In Atala A and Lanza R P, Eds. “Methods of Tissue Engineering”. Academic Press, San Diego, Calif. 2002; 203-218.
13. Ricordi C, Scharp D W, Lacy P E: Reversal of diabetes in nude mice after transplantation of fresh and 7-day-cultured (24 degrees C.) human pancreatic islets. Transplantation. 1988; 45(5):994-996.
14. FDA Biological Response Modifier Advisory Committee summary minutes Meeting #36, Oct. 9-10, 2003 [article online]. Available from Food and Drug Administration website http://www.fda.gov/ohrms/dockets/ac/cber03.html#BiologicalResponseModofiers
15. Weber D J, McFarland R D, Irony I: Selected Food and Drug Administration review issues for regulation of allogeneic islets of Langerhans as somatic cell therapy. Transplantation. 2002; 74(12):1816-1820.
16. Kuroda Y, Kawamura T, Suzuki Y, Fujiwara H, Yamamoto K, Saitoh Y: A new, simple method for cold storage of the pancreas using perfluorochemical. Transplantation. 1988; 46(3):457-460.
17. Ricordi C, Lacy P E, Finke E H, Olack B J, Scharp D W: Automated method for isolation of human pancreatic islets. Diabetes. 1988; 37(4):413-420.
18. Lukowiak B, Vandewalle B, Riachy R, Kerr-Conte J, Gmyr V, Belaich S, Lefebvre J, Pattou F: Identification and purification of functional human beta-cells by a new specific zinc-fluorescent probe. J Histochem Cytochem. 2001; 49(4):519-528.
19. Scaduto R C Jr, Grotyohann L W: Measurement of mitochondrial membrane potential using fluorescent rhodamine derivatives. Biophys J. 1999; 76(1 Pt 1):469-477.
20. Akao M, Teshima Y, Marban E: Antiapoptotic effect of nicorandil mediated by mitochondrial atp-sensitive potassium channels in cultured cardiac myocytes. J Am Coll Cardiol. 2002; 40(4):803-810.
21. Ricordi C, Kneteman N M, Scharp D W, Lacy P E: Transplantation of cryopreserved human pancreatic islets into diabetic nude mice. World J Surg. 1988; 12(6):861-865.
22. Ranuncoli A, Cautero N, Ricordi C, Masetti M, Molano R D, Inverardi L, Alejandro R, Kenyon N S: Islet cell transplantation: in vivo and in vitro functional assessment of nonhuman primate pancreatic islets. Cell Transplant. 2000; 9(3):409-414.
23. Keymeulen B, Anselmo J, Pipeleers D: The length of methabolic normalization after rat cell transplantation depends on endocrine cell composition of graft and on donor age. Diabetologia. 1997; 40(10):1152-1158.
24. Keymeulen B, Korbutt G, De Paepe M, Gorus F, Kloppel G, Pipeleers D G: Long term metabolic control by rat islet grafts depends on the composition of the implant. Diabetes. 1996; 45(12):1814-1821.
25. Kaneto H, Fujii J, Seo H C, Suzuki K, Matsuoka T, Nakamura M et al. Apoptotic cell death triggered by nitric oxide in pancreatic beta-cells. Diabetes. 1995; 44(7):733-738.
26. Wacker T, Jahr H, Weinand S, Brandhorst H, Brandhorst D, Lau D et al. Different toxic effects of hydrogen peroxide, nitric oxide, and superoxide on human, pig, and rat islets of Langerhans. Exp Clin Endocrinol Diabetes. 1995; 103 Suppl 2:133-135.
27. Delaney C A, Pavlovic D, Hoorens A, Pipeleers D C, Eizirik D L: Cytokines induce deoxyribonucleic acid strand breaks and apoptosis in human pancreatic islet cells. Endocrinology. 1997; 138(6):2610-2614.
28. Sweet I R, Khalil G, Wallen A R, Steedman M, Schenkman K A, Reems J A et al. Continuous measurement of oxygen consumption by pancreatic islets. Diabetes Technol Ther. 2002; 4(5):661-672.
29. Sweet I R, Cook D L, DeJulio E, Wallen A R, Khalil G, Callis J et al. Regulation of ATP/ADP in pancreatic islets. Diabetes. 2004; 53(2):401-409.
30. Ishii S, Sato Y, Terashima M, Saito T, Suzuki S, Murakami S et al. A novel method for determination of ATP, ADP, and AMP contents of a single pancreatic islet before transplantation. Transplant Proc. 2004; 36(4):1191-1193.
31. Zhang H J, Ansite J, Ihm S H, Oberbroeckling J, Friberg A, Hering B J. Number of islet beta cells better predicts sustained insulin-independence than number of islet equivalents (IE) transplanted in type 1 diabetic patients. Am J Transplant. 2004; 801 Suppl. 8:376.
32. Street C N, Lakey J R, Shapiro A M, Imes S, Rajotte R V, Ryan E A, Lyon J G, Kin T, Avila J, Tsujimura T, Korbutt G S. Islet graft assessment in the edmonton protocol: implications for predicting long-term clinical outcome. Diabetes. 2004; 53(12):3107-3114.

What is claimed is:

1. A method of assessing cellular composition and fractional viability that can be predictive of post-transplant cell potency and transplantation outcome, comprising:
identifying cellular composition and assessing cellular viability comprising
isolating cells from an organ, tissue;
dissociating the organ or tissue into single cells;
fixing, incubating with antibodies and/or staining of the single cells;
subjecting one aliquot of cells to laser scanning cytometry, immuno-histochemistry or electron microscopy; and,
subjecting one aliquot of cells to flow cytometry; and,
assessing cellular viability to predict transplantation outcome.

2. The method of claim 1, wherein the cells are stained with DNA and/or zinc binding dyes, 7-aminoactinomycin D (7-AAD), Fluorescein Diacetate, Ethidium Bromide or equivalent DNA binding stains.

3. The method of claim 1, wherein the cells are further stained with mitochondrial stains to assess cellular viability.

4. The method of claim 1, wherein the cells are further stained for apoptotic markers.

5. The method of claim 2, wherein the mitochondrial stains comprise: Newport Green PDX acetoxymethylether (NG); tetramethylrhodamine ethyl ester (TMRE), cyanine or xanthylium dyes.

6. The method of claim 1, wherein viable cells are 7-aminoactinomycin D negative (7-AAD⁻).

7. The method of claim 1, wherein the cells are isolated from the pancreas.

8. The method of claim 1, wherein the cell composition comprises beta cells, alpha cells, acinar cells and ductal cells.

9. The method of claim 7, wherein the beta cells are NG^{bright}TMRE⁺.

10. The method of claim 1, wherein antibodies are specific for pancreatic cell markers and subsets thereof, comprising insulin, glucagon, somatostatin, pancreatic polypeptide, ductal cell markers, progenitor or stem cell markers, inflammatory or immune cell markers.

11. The method of claim 1, wherein assessing cellular viability and predictive transplantation outcome comprises quantifying cellular composition and fractional beta-cell viability.

12. The method of claim 10, wherein the cellular composition is quantified by measuring viable β-cell index, β-cell Mass/kg, Viable β-cell Mass/kg.

13. The method of claim 10, wherein the predictive transplantation outcome is measured as:
Diabetes Reversal Index=total islet equivalents (IEQ)×(% β-cell content in the islets)×(% non-apoptotic β-cells)/Insulin IU×10,000.

14. The method of claim 10, wherein Viable β-cell Equivalent Number/kg (vβEQ/kg)=[islet β-cell content×β-cell fractional viability×IEQ/kg] and compared to insulin reduction rate per kg, and insulin independence after islet infusion into a patient.

15. A method of identifying cells suitable for transplantation comprising;
isolating cells from an organ, tissue or bodily fluids;
identifying cellular composition;
assessing viability of cells in the cellular composition; and,
identifying cells suitable for transplantation.

16. The method of claim 14, wherein specific cells are isolated and viability is determined.

17. The method of claim 14, wherein specific cells are identified by cell specific antigens, biomarkers, antibodies and functional assays.

18. The method of claim 14, wherein cellular composition and viability are assessed by laser scanning cytometry and cytofluorimetry.

19. A method of identifying cell damage comprising:
isolating cells from an organ, tissue or bodily fluids;
identifying cellular composition;
assessing viability of cells in the cellular composition; and,
identifying cell damage.

20. A method of identifying ductal cells and determining viability and/or potency, comprises:
identifying cellular composition and assessing cellular viability comprising
isolating cells from an organ, tissue;
dissociating the organ or tissue into single cells;
fixing, incubating with antibodies and/or staining of the single cells;
subjecting one aliquot of cells to laser scanning cytometry, immuno-histochemistry or electron microscopy; and,
subjecting one aliquot of cells to flow cytometry,
assessing cellular viability and/or potency.

21. The method of claim 20, wherein ductal cells are identified by pan-ductal membrane antibody, CA19-9.

22. The method of claim 20, wherein cellular composition is assessed by laser scanning cytometry.

23. The method of claim 20, wherein identification of ductal cells is predictive for long term function.

24. A method of identifying of progenitor and/or stem cells, and determining their viability/potency, predictive of the regenerative potential and/or long term function, comprising:
isolating cells from bone marrow or organ;
dissociating the bone marrow or organ into single cells;
fixing, incubating with antibodies and/or staining of the single cells;
subjecting one aliquot of cells to laser scanning cytometry, immuno-histochemistry or electron microscopy; and,
subjecting one aliquot of cells to flow cytometry
identifying of progenitor and/or stem cells, and determining their viability/potency, predictive of the regenerative potential and/or long term function.

25. The method of claim 24, wherein stem cells are identified by stem cell markers.

26. The method of claim 24, wherein cellular composition is assessed by laser scanning cytometry.

27. A method of identifying and determining the viability/potency of inflammatory and immune cells predictive of the early loss of transplanted cells after infusion/implantation and/or predictive of the probability of acute and chronic rejection/recurrence of autoimmunity, comprising:
isolating cells from organ, tissue or bodily fluid;
obtaining single cells from the organ tissue, or bodily fluid;
fixing, incubating with antibodies and/or staining of the single cells;
subjecting one aliquot of cells to laser scanning cytometry, immuno-histochemistry or electron microscopy; and,
subjecting one aliquot of cells to flow cytometry; and,
identifying and determining the viability/potency of inflammatory and immune cells predictive of the early loss of transplanted cells after infusion/implantation and/or predictive of the probability of acute and chronic rejection/recurrence of autoimmunity.

28. The method of claim 27, wherein inflammatory and immune cells are identified by cell specific markers comprising: MCP-1, HLA Class I and II, CD80, CD86, CD40, CD40L, TGF-beta, interleukins, α, β, or γ-IFN, TNF, CD4, CD25, Foxp3, VEGF receptor-2(FLK-1), TRK (an NGF receptor), transferrin receptor, and annexin II (lipocortin 2), CD4, CD104, CD117, heat shock protein-27, tumor rejection antigen, glutathione-S transferase, peroxiredoxin 1, voltage-dependent-anion channel-2, protein kinase C substrate, phosphatase 2A inhibitor, esterase D, RNase A, initiation factor 5a, elongation factor 1-alpha, ribosomal protein S12, ribosomal protein large P1, ribosomal protein large P2, transcription factor BTF 3a, annexin I, destrin, myosin light chain, lactate dehydrogenase A, glycerolaldehyde-3-P dehydrogenase, citrate synthetase, transketolase, P-glycerolmutase, aldo-keto reductase 7(A2), alpha-amylase inhibitor CM3, enoyl-CoA hydratase, proteosome subunit alpha-4, stromal derived factor 1 (SDF-1), MCP-1, MIP-1α, MIP-1β, RANTES, exotaxin IL-8, C3a, P-selectin, E-selectin, LFA-1, VLA-4, VLA-5, CD44, MMP activation, VEGF, EGF, PDGF, VCAM, ECAM, G-CSF, GM-CSF, SCF, EPO, tenascin, MAdCAM-1, α4 integrins, α5 integrins, beta defensins 3 and 4, annexin V, TUNEL Stain, 7-amino-actinomycin D and Caspase substrates.

29. The method of claim 27, wherein cellular composition is assessed by laser scanning cytometry.
